(19) United States
(12) Patent Application Publication
(10) Pub. No.: US 2016/0353757 A1
(43) Pub. Date: Dec. 8, 2016

(54) Shell Egg Pasteurization Processes

(71) Applicant: National Pasteurized Eggs, Inc., Lansing, IL (US)

(72) Inventors: Hector Gregorio Lara, Plainfield, IL (US); Erdogan Ceylan, Crete, IL (US)

(73) Assignee: National Pasteurized Eggs, Inc., Lansing, IL (US)

(21) Appl. No.: 15/170,590

(22) Filed: Jun. 1, 2016

Related U.S. Application Data
(60) Provisional application No. 62/169,740, filed on Jun. 2, 2015.

(51) Int. Cl. A23B 5/005; G01N 33/08

(52) U.S. Cl. CPC A23B 5/0052; G01N 33/08; A23V 2002/00

(57) ABSTRACT

The D-value for pasteurizing chicken shell eggs that are not refrigerated prior to pasteurization, or shell eggs that are sufficiently tempered to room temperature prior to pasteurization, is less than the D-value for pasteurizing chicken shell eggs that are refrigerated prior to pasteurization. Statistically verified time and temperature pasteurization protocols that produce pasteurized chicken shell eggs to achieve at least a statistical 5-log reduction of Salmonella Enteritidis are therefore possible at lower pasteurization temperatures or lower pasteurization dwell times.

### FIELD OF THE INVENTION

The invention relates to shell egg pasteurization processes. In particular, the invention pertains to statistically verified time and temperature pasteurization protocols that produce pasteurized chicken shell eggs to achieve at least a statistical 5-log reduction of Salmonella Enteritidis.

### BACKGROUND OF THE INVENTION

The Assignee of the present application owns several patents pertaining to the pasteurization of shell eggs including, for example, U.S. Pat. No. 6,165,538 entitled “Pasteurized In-Shell Chicken Eggs”, by Leon John Davidson, issuing on Dec. 26, 2000; U.S. Pat. No. 6,113,961 entitled “Apparatus and Methods for Pasteurizing in-Shell Eggs,” issuing on Sep. 5, 2000, by Louis Polster, and U.S. Pat. No. 9,289,002, entitled “Shell Egg Pasteurization Method” issuing on Mar. 22, 2016 by Hector Lara, each of which is incorporated herein by reference. Current commercial pasteurization processes heat batches of shell eggs in heated water and/or humid air. In the process discussed in the Lara '002 patent, batches of shell eggs are submerged in a heated water bath and are moved sequentially from stage to stage in order to complete the pasteurization process. The present invention relates to the thermal treatment necessary for sufficient pasteurization. While other pasteurization systems may not move the batches of eggs sequentially through zones in a water bath, certain aspects of the present invention may apply to those other systems as well.

The purpose of the pasteurization process is to heat the shell egg such that the entire egg including the center of the egg yolk warms to an adequate temperature for a sufficient amount of time to meet or exceed the accepted standard for reduction of Salmonella Enteritidis set by the FDA. A 5-log reduction of Salmonella Enteritidis is the regulated standard set by the FDA (Food and Drug Administration) for pasteurization of in-shell chicken eggs.

It is critical that sufficient heat be provided to meet the 5-log kill standard throughout the entire mass of the egg; however, it is also important the egg not be overheated during the pasteurization process. Overheating can result in partial cooking or loss of quality and functionality of the shell egg. There are many characteristics pertaining to egg quality and functionality, see for instance the characteristics measured and observed in the above incorporated Davidson '538 patent. One of the most common functionality tests is to measure albumen quality in Haugh units. As an unpasteurized egg ages, the thick inner portion of the albumen tends to thin. Haugh units are calculated using both the egg weight and the height of the inner thick albumen of an egg cracked open on a flat surface. Standard Haugh unit values for different grades of eggs are follows: Grade AA is greater than 72 Haugh units, Grade A is between 60 and 72 Haugh units, and Grade B is less than 60 Haugh units. The USDA (United States Department of Agriculture) requires that all eggs for human consumption be graded both in terms of weight (minimum weight requirements for applicable size e.g.: Medium, Large and Extra-Large) and quality as measured in Haugh units (Grade AA, Grade A, Grade B). It is known in the art, however, that pasteurization leads to higher Haugh unit values compared to a corresponding unpasteurized egg. During the pasteurization process, thermal energy causes the albumen to denature and then cross link, which results in a higher tighter inner albumen and higher Haugh unit values. As more heat is added, the albumen becomes cloudy and eventually begins to coagulate as well. The Davidson '538 patent recognizes that increased Haugh unit values and cloudy egg whites occur with pasteurization. It is also recognized in the art that the time to whip albumen to peak height increases about 8-fold when an egg is pasteurized using current thermal techniques. A goal of the present invention is to use a gentler, statistically verifiable process that reliably achieves a 5-log kill of Salmonella Enteritidis without causing as much cloudiness in the albumen as current thermal techniques.

In prior art batch processing pasteurization equipment using a heated water bath, each batch contains many dozens of eggs typically arranged in flats and stacked one upon another, for example, as described in the incorporated Polster '961 patent and the Lara '002 patent. Prior to pasteurization, the stacks of eggs are staged and held at a uniform start temperature. For example, refrigerated stacks of eggs may be held at 45° F. for storage and then moved to and placed into the pasteurization bath with an egg start temperature of 45° F. Alternatively, refrigerated or unrefrigerated eggs may be tempered to room temperature, e.g., 65° F., prior to being moved to and placed in the pasteurization bath. The batch processing control system is programmed with pasteurization protocols that set water bath temperature and overall dwell time depending on the egg size (e.g., Medium size versus Large size) and start temperature for the batch. Significant efforts have been made in the art to reliably heat pasteurized shell eggs to consistently achieve the required, accumulated 5-log kill without overcooking the eggs, see e.g., Schuman et al., “Immersion heat treatments for inactivation of Salmonella Enteritidis with intact eggs,” Journal of Applied Microbiology 1997, 83, 438-444, and the incorporated Davidson '538 patent.

A D-value (measured in minutes) is the amount of time that it takes to achieve a log kill of a pathogen (e.g., Salmonella Enteritidis) in a substance held at a certain temperature. D-values for Salmonella Enteritidis are known to be higher in egg yolk than in albumen, which means that it is more difficult to kill Salmonella Enteritidis in egg yolk than in albumen. The Davidson '538 patent is based in part on the notion that heating a shell egg in a water bath requires heat to transfer through the shell and through the albumen to the yolk, so the temperature of the albumen will necessarily be greater than the temperature of the yolk when the egg is coming up to the temperature of the water bath. According to FDA requirements and the Davidson '538 patent, the yolk temperature must be at least 128° F. before Salmonella Enteritidis is killed reliably. The Davidson '538 patent therefore provides a statistically derived line plotting a 5 D-value (i.e., five times the D-value) for shell eggs inoculated with Salmonella Enteritidis having yolk temperatures from 128° F. to 138° F. As the egg yolk heats from 128° F. to the water bath temperature (“come up time”), the log kill accumulates and it continues to accumulate as the yolk is maintained at or near the water bath temperature. In fact, log kill would continue to accumulate even after the shell egg is removed from the pasteurization bath until the yolk temperature drops below 128° F. The Davidson '538 patent teaches cooling the pasteurized eggs rapidly after the eggs are removed from the pasteurization bath in order to avoid further denaturation of the albumen.

In the system described in the Lara '002 patent, each batch of eggs is held in a carrier that is supported by a gantry located above the water bath and is moved in stages through the water bath. An advance motor moves the respective carriers sequentially from stage to stage at fixed time intervals. A heating system heats the water bath to a temperature set point in accordance with the pasteurization protocol selected for the size and start temperature of the batches being pasteurized. Pressurized air is supplied through openings in air supply tubes into the water bath to cause perturbation and facilitate effective, uniform heat transfer throughout the stacks of shell eggs. The level of the air flow can also be set in the pasteurization protocols as disclosed in the Lara '002 patent. Since there is a risk of temperature spikes occurring in the pasteurization bath that can cause overcooking and poor quality pasteurized eggs, the system in the Lara '002 patent provides a cooling system for the pasteurization bath. The pasteurization protocol as taught in the Lara '002 patent is programmed with an upper temperature limit that is higher than the temperature set point for the heating system. The cooling system operates to lower the temperature of the water bath as the temperature in the bath approaches the upper temperature limit and in turn mitigates any temperature spikes. The use of a cooling system in this manner enables the pasteurization system to aggressively maintain the water bath temperature at or near the minimum required temperature for the 5-log time and temperature protocol.

After removal from the pasteurization bath, the eggs are sprayed with an antibacterial agent, and coated with food-grade wax or other sealant to protect the eggs from outside contaminants and improve shelf life. The eggs are also marked on the shells to designate the eggs have been pasteurized. Despite the close attention and effort to not overcook and otherwise maintain high quality and functionality standards, those in the art are continually searching for ways to improve the quality and functionality of pasteurized in-shell chicken eggs.

### SUMMARY OF THE INVENTION

The inventors have discovered that the D-value for Salmonella Enteritidis in the yolk of in-shell chicken eggs is substantially lower for in-shell chicken eggs that have not been refrigerated after being laid prior to pasteurization (or have been suitably tempered to room temperature prior to pasteurization), compared to shell eggs that are refrigerated prior to pasteurization. More specifically, the D-value in the Davidson '538 patent at 133° F., for example, is approximately ten minutes. A D-value of about ten minutes (see Table 3 below, rows 1 and 2) was confirmed by the inventors running tests on shell eggs stored at a refrigerated temperature of 45° F. prior to pasteurization. However, identical tests run on unrefrigerated eggs tempered to 65° F., resulted in a D-value within the range of 6.42 to 7.26 minutes. This lower D-value means that pasteurization of in-shell chicken eggs in a water bath held at 133° F. achieves the required 5-log kill of Salmonella Enteritidis in the yolk in about 9 to 10 minutes less than what was previously thought necessary. In other words, an accumulated 5-log kill for a room temperature, unrefrigerated Large egg placed in a water bath held at 133° F. is achieved in about 50 minutes. Also, a 5-log reduction when pasteurizing Large shell eggs in a water bath held at 134° F. can be accomplished in less time than in the prior art as well, e.g. in about 45 minutes. The shorter dwell time in a 133° F. or 134° F. (or higher temperature) water bath means that the albumen may be affected less by the pasteurization process and may closer resemble the quality and functionality of an unpasteurized egg than the albumen of an egg pasteurized using the protocols previously deemed necessary to achieve a 5-log reduction of Salmonella Enteritidis in the prior art. The shorter dwell times also mean higher equipment throughput can be achieved for a given water bath temperature. Dwell times can be shortened by a few minutes even when the prior art pasteurization models are used by starting with the eggs tempered to room temperature (e.g., 65° F.) rather than starting with eggs that are held at a refrigerated temperature (e.g. 45° F.), simply because there is a shorter come up time for the yolk temperature to reach the temperature of the water bath. The invention, however, capitalizes on the discovery that the D-value for Salmonella Enteritidis in the yolk of an in-shell chicken egg that has not been refrigerated prior to pasteurization (or has been suitably tempered to room temperature prior to pasteurization) is lower compared to the D-value for the yolk of shell eggs that are refrigerated prior to pasteurization by creating a D-value pasteurization model that predicts the reduction of Salmonella Enteritidis in the yolk of the chicken eggs held at an unrefrigerated temperature prior to pasteurization. Because the D-values are less in the pasteurization model, the required dwell times are shortened beyond the dwell times required to pasteurize eggs tempered to room temperature (e.g. 65° F.) using the prior art D-value pasteurization models disclosed in the Davidson '538 patent or by Schuman et al.

In one aspect, the invention is directed to a shell egg pasteurization method that involves heating a water bath to a temperature set point, e.g. about 133° F. or 134° F., tempering unrefrigerated shell eggs to room temperature, e.g. 65° F., or tempering refrigerated shell eggs to room temperature (e.g. 65° F.) for a suitable length of time, and then placing the tempered shell eggs in the heated water bath. Typically, a stack of flats of chicken eggs each having the same size and each being tempered to room temperature are placed in the bath together as a batch. In accordance with the invention, an empirically verified D-value pasteurization model is determined for the statistical reduction of Salmonella Enteritidis in the yolk of chicken shell eggs held at an unrefrigerated temperature prior to pasteurization. If the eggs were refrigerated, tempering the eggs for 3 days at room temperature has been found suitable to obtain the reduced D-values. The chicken shell eggs are held in the water bath for a dwell time calculated by the D-value pasteurization model to achieve at least a 5-log reduction of Salmonella Enteritidis that may be present in the yolk. At the end of the calculated dwell time, the egg or batch of eggs are pulled from the heated water bath.

In one embodiment, a shell egg pasteurization system implementing the invention includes a pasteurization water bath having a series of stages through which the batches of shell eggs are moved. Temperature sensors measure the water temperature in the bath. A heating system operates to heat the temperature of the water uniformly throughout the bath to the temperature set point value. The heating system includes many independently controlled heating elements spanning over the floor of the bath to achieve uniform heating throughout the bath. A batch carrier arrangement holds batches of shell eggs in the bath and includes an advance motor that moves the batch carrier arrangement and the respective batches of shell eggs through and between stages in the bath. It is desirable that the batches of eggs be pasteurized in stacks of eggs on flats. It is further desirable that air bubbles flow into the water bath under the heating elements and the stacks of eggs to facilitate even heating of all the eggs in the stack.

The system also includes a batch processing control system that is programmed with at least one pasteurization protocol. In accordance with the invention, the pasteurization protocol is based on the batch of eggs being unrefrigerated and tempered to room temperature. For example, the protocol may set the temperature set point value for the water bath at about 133° F., and the total dwell time for each batch in the bath to a predetermined time of 50 minutes for Large sized eggs having a start temperature of 65° F. As mentioned, the predetermined dwell time must be sufficient to ensure that both the yolk and albumen of the eggs are pasteurized to achieve a 5-log reduction of Salmonella Enteritidis that may be present in the yolk and albumen of the eggs. As discussed above, the D-value for Salmonella Enteritidis in yolk at desirable pasteurization temperatures when the eggs are unrefrigerated has unexpectedly been found to be substantially less than the D-value when the eggs are refrigerated.

The D-value in the exemplary embodiments has been calculated based on room temperature eggs held at 65° F.; however, the D-value testing protocol can be carried out in accordance with the invention at temperatures higher or lower than 65° F. that are considered to be room temperature, such as up to 85° F. for tropical climates. Above 85° F., there is a likelihood that bacterial growth will occur in the eggs and therefore it is not advised to hold the eggs above 85° F. prior to pasteurization.

It has also been confirmed that Salmonella on the surface of chicken shell eggs is reduced by 5-log in 10 minutes or less when the eggs are placed in a 133° F. water bath.

### BRIEF DESCRIPTION OF THE DRAWINGS

FIG. 1 is a schematic drawing illustrating the movement of batches of eggs through multiple stages in an exemplary shell egg pasteurization system.

FIG. 2 is a view taken along line 2-2 in FIG. 1.

FIG. 3 schematically illustrates a time and temperature control system constructed in accordance with the invention for use in the exemplary shell egg pasteurization system of FIG. 1.

FIG. 4 is a schematic illustration of a PID algorithm used to individually control heating elements in accordance with the exemplary embodiment of the invention.

FIG. 5A is a plot of data pertaining to the inactivation of Salmonella in Large eggs stored and refrigerated at 45° F. and pasteurized at 133° F. (±0.5° F.)—Lot 1.

FIG. 5B is a plot of data pertaining to the inactivation of Salmonella in Large eggs stored and refrigerated at 45° F. and pasteurized at 133° F. (±0.5° F.)—Lot 2.

FIG. 5C is a plot of data pertaining to the inactivation of Salmonella of Large shell eggs stored at 45° F. tempered to 65° F. and pasteurized at 133° F. (±0.5° F.)—Lot 1.

FIG. 5D is a plot of data pertaining to the inactivation of Salmonella of Large shell eggs stored at 45° F. tempered to 65° F. and pasteurized at 133° F. (±0.5° F.)—Lot 2.

FIG. 5E is a plot of data regarding the inactivation of Salmonella unrefrigerated, Large shell eggs tempered to 65° F. and pasteurized at 133° F. (±0.5 F)—Lot. 1.

FIG. 5F is a plot of data regarding the inactivation of Salmonella unrefrigerated, Large shell eggs tempered to 65° F. and pasteurized at 133° F. (±0.5 F)—Lot. 2.

FIG. 6A is a plot of data pertaining to the inactivation of Salmonella in Medium eggs stored and refrigerated at 45° F. and pasteurized at 133° F. (±0.5° F.)—Lot 1.

FIG. 6B is a plot of data pertaining to the inactivation of Salmonella in Medium eggs stored and refrigerated at 45° F. and pasteurized at 133° F. (±0.5° F.)—Lot 2.

FIG. 6C is a plot of data pertaining to the inactivation of Salmonella of Medium shell eggs stored at 45° F. tempered to 65° F. and pasteurized at 133° F. (±0.5° F.)—Lot 1.

FIG. 6D is a plot of data pertaining to the inactivation of Salmonella of Medium shell eggs stored at 45° F. tempered to 65° F. and pasteurized at 133° F. (±0.5° F.)—Lot 2.

FIG. 6E is a plot of data regarding the inactivation of Salmonella unrefrigerated, Medium shell eggs tempered to 65° F. and pasteurized at 133° F. (±0.5 F)—Lot. 1.

FIG. 6F is a plot of data regarding the inactivation of Salmonella unrefrigerated, Medium shell eggs tempered to 65° F. and pasteurized at 133° F. (±0.5 F)—Lot. 2.

FIG. 7A is a plot of time and temperature data for the center of the yolk of Large shell eggs that have been stored and refrigerated at 45° F. and pasteurized at 133° F. (±0.5° F.)—Lot 1.

FIG. 7B is a plot of time and temperature data for the center of the yolk of Large shell eggs that have been stored and refrigerated at 45° F. and pasteurized at 133° F. (±0.5° F.)—Lot 2.

FIG. 7C is a plot of data pertaining to the temperature for the center of the yolk of Large shell eggs that are refrigerated at 45° F., tempered to 65° F. before pasteurization and pasteurized at 133° F. (±0.5° F.)—Lot. 1.

FIG. 7D is a plot of data pertaining to the temperature for the center of the yolk of Large shell eggs that are refrigerated at 45° F., tempered to 65° F. before pasteurization and pasteurized at 133° F. (±0.5° F.)—Lot. 2.

FIG. 7E is a plot of data pertaining to the temperature for the center of the yolk of unrefrigerated Large shell eggs, tempered to 65° F. and pasteurized at 133° F. (±0.5° F.)—Lot 1.

FIG. 7F is a plot of data pertaining to the temperature for the center of the yolk of unrefrigerated Large shell eggs, tempered to 65° F. and pasteurized at 133° F. (±0.5° F.)—Lot 2.

FIG. 8A is a plot of time and temperature data for the center of the yolk of Medium shell eggs that have been stored and refrigerated at 45° F. and pasteurized at 133° F. (±0.5° F.)—Lot 1.

FIG. 8B is a plot of time and temperature data for the center of the yolk of Medium shell eggs that have been stored and refrigerated at 45° F. and pasteurized at 133° F. (±0.5° F.)—Lot 2.

FIG. 8C is a plot of data pertaining to the temperature for the center of the yolk of Medium shell eggs that are refrigerated at 45° F., tempered to 65° F. before pasteurization and pasteurized at 133° F. (±0.5° F.)—Lot. 1.

FIG. 8D is a plot of data pertaining to the temperature for the center of the yolk of Medium shell eggs that are refrigerated at 45° F., tempered to 65° F. before pasteurization and pasteurized at 133° F. (±0.5° F.)—Lot. 2.

FIG. 8E is a plot of data pertaining to the temperature for the center of the yolk of unrefrigerated Medium shell eggs tempered to 65° F. and pasteurized at 133° F. (±0.5° F.)—Lot 1.

FIG. 8F is a plot of data pertaining to the temperature for the center of the yolk of unrefrigerated Medium shell eggs tempered to 65° F. and pasteurized at 133° F. (±0.5° F.)—Lot 2.

FIG. 9A is a plot showing the inactivation of Salmonella in unrefrigerated Large shell eggs pasteurized at 131° F. (±0.5° F.)—Trial 1.

FIG. 9B is a plot showing the inactivation of Salmonella in unrefrigerated Large shell eggs pasteurized at 131° F. (±0.5° F.)—Trial 2.

FIG. 9C is a plot showing the inactivation of Salmonella in unrefrigerated Large shell eggs pasteurized at 131° F. (±0.5° F.)—Trial 3.

FIG. 10 is a plot showing the inactivation of Salmonella in unrefrigerated Large shell eggs pasteurized at 134° F. (±0.5° F.).

FIG. 11 is a plot showing the inactivation of Salmonella in unrefrigerated Large shell eggs pasteurized at 135° F. (±0.5° F.).

FIG. 12A is a plot of data pertaining to the temperature for the center of the yolk of unrefrigerated Large shell eggs pasteurized at 131° F. (±0.5° F.)—Trial 1.

FIG. 12B is a plot of data pertaining to the temperature for the center of the yolk of unrefrigerated Large shell eggs pasteurized at 131° F. (±0.5° F.)—Trial 2.

FIG. 12C is a plot of data pertaining to the temperature for the center of the yolk of unrefrigerated Large shell eggs pasteurized at 131° F. (±0.5° F.)—Trial 3.

FIG. 13 is a plot of data pertaining to the temperature for the center of the yolk of unrefrigerated Large shell eggs pasteurized at 134° F. (±0.5° F.).

FIG. 14 is a plot of data pertaining to the temperature for the center of the yolk of unrefrigerated Large shell eggs pasteurized at 135° F. (±0.5° F.).

FIG. 15 is a regression plot of measured D-values used to calculate the z-value of Salmonella in unrefrigerated Large shell eggs.

### DETAILED DESCRIPTION OF THE DRAWINGS

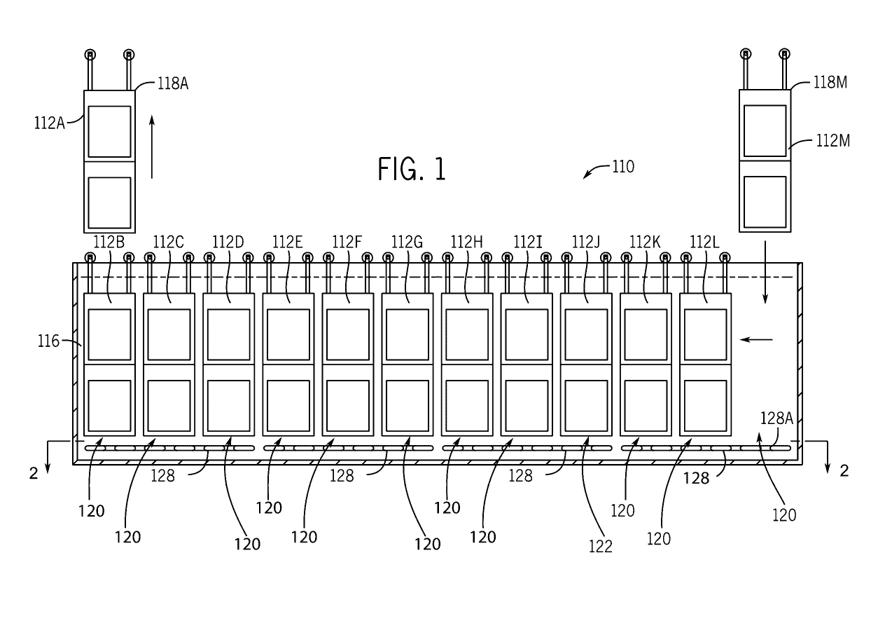

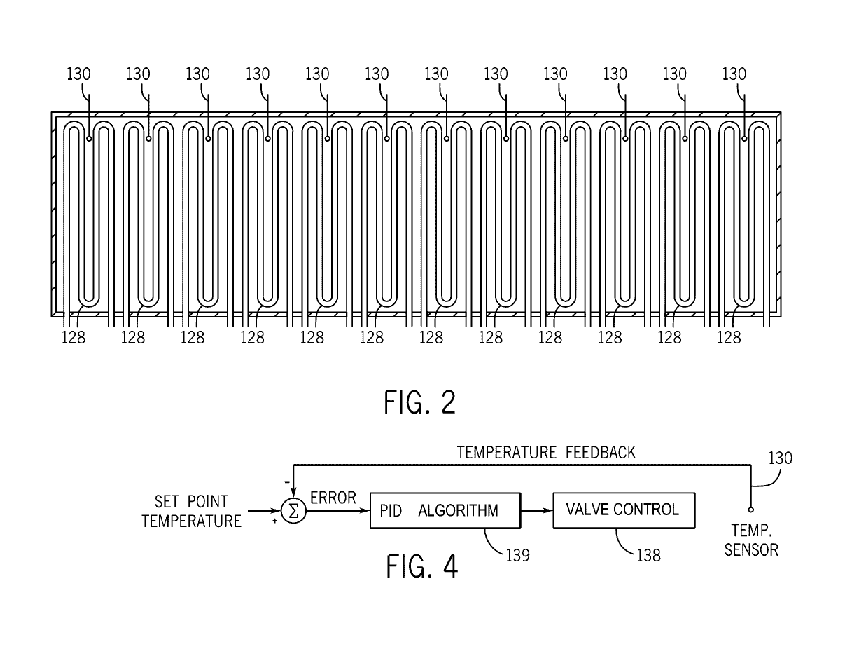

FIGS. 1 and 2 illustrate a shell egg pasteurization system 110 in which batches 112A through 112M of eggs are passed in stages through a heated pasteurization water bath 116. In commercial operations, it is not efficient or cost effective to pasteurize a single egg, a single row or even a single layer of shell eggs at a time. Therefore, it is known in the art to pasteurize batches of eggs containing several stacked layers of eggs. For example, a flat may contain 2½ dozen eggs, with a stack containing four layers of flats and each batch containing 16 stacks loaded onto a carrier 118A through 118M. Generally speaking, the bath 116 must heat not only the eggs, but also the flats and the carriers. Even though the carriers 118A-118M and the flats are typically the same whether the eggs being pasteurized are Medium size, Large, Ex-Large or Jumbo, the different size and weight of the eggs will normally impact the heat required in different parts of the bath 116 differently.

As shown in FIG. 1, in the pasteurization bath 116 includes twelve (12) staging positions 120. FIG. 1 also shows a carrier 118M containing batch 112M of eggs prior to being placed in the bath 116, and carrier 118A containing batch 112A of eggs which has been removed from the bath at 116 after pasteurization. In the exemplary embodiment of the invention, the batch 112M of eggs are tempered at 65° F. prior to being placed in the bath 116. In addition, the eggs 112M are either unrefrigerated after being laid, or are tempered for suitable amount of time, e.g. 3 days at room temperature. The D-value pasteurization model calculated below under Example 2 is suitable for chicken shell eggs that are unrefrigerated or have been held at room temperature for a sufficient amount of time, e.g. 3 days, prior to pasteurization. Note that the carriers 118B through 118L containing batches of eggs 112B through 112L are located within the bath 116 and have been moved forward one stage in preparation for the bath 116 to receive carrier 118M and batch 112M in the first staging position 120. The heated water in the bath 116 is allowed to flow between staging positions within the bath. It should be understood, however, that the schematic representation of the system 110 in FIG. 1 is merely representative of a type of pasteurization system in which the invention may be used, and that the invention may be useful with other types of pasteurization systems.

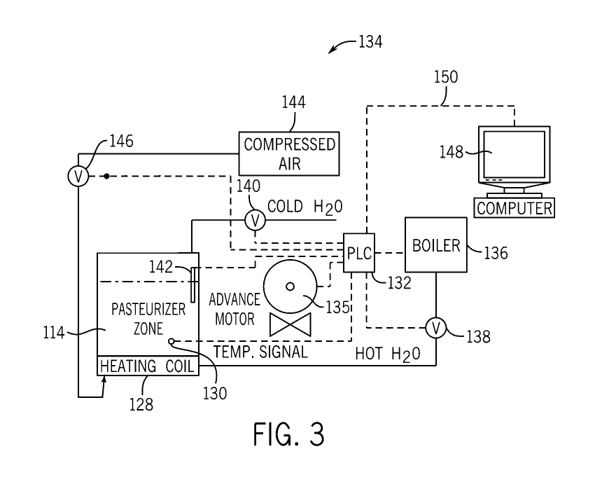

Referring still to FIGS. 1 and 2, sets of heating coils 128 are located in that water bath 116 to heat the fluid within the bath. In accordance with the exemplary embodiment of the invention, each of the heating elements 128 is controlled independently. Temperature sensors 130, such as RTDs, are located in the general vicinity of each respective heating element 128. While the invention can be implemented with a single temperature sensor associated with each heating element 128, one of ordinary skill in the art will understand that it is possible to use additional temperature sensors in the vicinity of the respective heating elements 128 for purposes of redundancy and averaging. It is also desirable to inject air bubbles into the water bath 116 below the heating elements 128 to facilitate even heating of the shell eggs in the respective stacks. The temperature sensors 130 are electrically connected to a programmable logic controller (PLC) 132, see FIG. 3.

FIG. 3 schematically illustrates the operation of a batch processing control system 134 to control both the temperature of the water in the bath 116 near the respective heating elements 128 as well as the movement of the advance motor 135 to advance the batches 112A, 112M from stage to stage in the bath 116. The PLC 132 is programmed in accordance with a predetermined pasteurization protocol for batches of eggs having the designated size and start temperature.

The PLC 132 preferably contains uploaded software in a machine readable form on a data storage device or in memory that is able to implement a plurality of predetermined pasteurization protocols, each being statistically verified, and each being customized for a distinct combination of egg size and start temperature, and in accordance with the invention whether the shell eggs have not been refrigerated and/or have been appropriately tempered to room temperature (e.g. 65° F., or generally at a room temperature in the range of about 60° F.-85° F.) in order to justify the use of a time and temperature protocol taking advantage of lower D-values. For example, the controller 132 may contain six formulas: one pair of formulas for full batches of Medium sized eggs with one for refrigerated eggs with a start temperature of 45° F. and the other for unrefrigerated eggs with a start temperature at room temperature; a second pair of formulas for full batches of Large sized eggs with one formula for refrigerated eggs with a start temperature of 45° F. and the other for unrefrigerated eggs with a start temperature at room temperature; and a third pair of formulas for full batches of extra-Large sized eggs again with one formula for refrigerated eggs with a start temperature of 45° F. and another formula for unrefrigerated eggs with a start temperature at room temperature. Each formula will likely have a unique dwell time, and possibly unique target temperatures.

The water bath target temperature is desirably 133° F. (±0.5° F.) or 134° F. (±0.5° F.). For example, the total dwell time for an unrefrigerated batch of Large eggs having a start temperature equal to room temperature (e.g. 65° F.) may be 50 minutes, which means that each batch of eggs spends very roughly slightly more than four (4) minutes in each stage position 120, FIG. 1, as the batch 112 moves through the pasteurizer 116. In the system 110 shown in FIGS. 1 and 2, individual batches 118M would be placed within the pasteurizer bath 116 every four minutes and 10 seconds according to this hypothetical protocol, and each batch 118A-L would remain in each stage 120 for four minutes and 10 seconds. Referring again to FIG. 3, the PLC is programmed to hold each batch of shell eggs 112A-112M in each of the respective staging position for the preselected period of time, and the PLC 132 will transmit a control signal to the motor advance 135 accordingly.

FIG. 3 shows the PLC 132 controlling one heating coil 128, although it should be understood that in the system illustrated in FIGS. 1 and 2 the PLC 132 will independently control each of the multiple heating elements 128 in the bath. As shown in FIG. 3, for each heating element 128, the PLC 132 receives a signal from at least one temperature sensor 130. In particular, an electronically controlled valve 138 controls the flow of heated water from the boiler 136 to the respective heating element 128. In other words, there is a separate electronically controlled valve 138 for each individual heating element 128. Referring to FIG. 4, the PLC uses a proportional-integral-derivative (PID) algorithm, block 139, to control the operation of each heating element 128 independently. For example, in the system shown in FIGS. 1 and 2, the PLC will be programmed with separate PID algorithms to control the operation of the multiple heating elements 128. The temperature of the water in the vicinity of the respective heating element 128 is continuously monitored by the respective temperature sensor 130. The loop time for the PID algorithm is preferably five seconds although other loop times may be used in accordance with the invention. PID algorithms are generally known in the art. The set point temperature value for the heating element 128 is defined by the predetermined pasteurization protocol. The difference between the set point temperature value and the temperature feedback signal from the temperature sensor 130 results in an error signal that drives the PID algorithm 139. The error signal drives the PID algorithm to generate a control signal that is transmitted to the valve control 138. The proportional aspect of the PID algorithm pertains to the severity of the error gap and uses a constant to calculate the amount of time the valve should be open to eliminate the gap. The integral aspect essentially measures how long the error gap has occurred, and the derivative aspect measures the rate of change of the proportional aspect. Each of these various aspects is combined to generate a control signal transmitted to the electronic valve 138 for each cycle of the loop. Preferably, the control signal is a value between zero and one and represents the percentage of time that the valve 138 will be open for the 5 second loop interval. For example, if the generated control signal from the PID algorithm is one, the valve will remain open to allow the flow of hot water to the heating element 128 for the entire 5 second cycle. On the other hand, if the generated control signal is only 0.8, the valve will be open for 4 out of 5 seconds. In this manner, the PLC 132 precisely controls the operation of each individual heating element 128, thereby preventing large swings in temperature.

A separate PID algorithm is used to control the temperature of the water supplied by the boiler 136, for example at about 170° F.

While the set point temperature value is a precise target value for the temperature of the bath in the vicinity of the respective heating element 128, the pasteurization protocol may also include an upper temperature limit, e.g. a half of degree Fahrenheit above or below the set point temperature value. The use of the multiple individually controlled heating elements is normally effective in maintaining the temperature of the water bath within the desired temperature range. If, however, the temperature in one or more areas of the pasteurization bath approaches the upper temperature limit, the PLC 132 will operate the cold water flow valve 140 to add cold water to the bath. Typically, there will only be one cold water valve, although there may be several. In any event, it is desirable that the operation of the cold water valve be controlled by a separate PID algorithm in the PLC, and if the system includes multiple cold water valves that each one be independently controlled.

The system also includes a level sensor 142 that senses the level of water in the pasteurizer. As in the prior art, if the water level drops below the location of the level sensor 142, the PLC 132 will add cold make up water by opening flow control valve 140. When this occurs, it will also normally be necessary for the controller 132 to control the hot water flow control valves 138 to provide hot water to the heating coils 128 in order to maintain the temperature of the bath within the accepted temperature range for the given protocol programmed on the PLC 132.

FIG. 3 also illustrates a compressed air source 144 along with a control valve 146 to control the level of compressed air being supplied to produce air bubbles throughout the pasteurizer bath 114. The PLC 132 can optionally control the flow control valve 146 for the compressed air in accordance with the predetermined pasteurization protocol programmed on the PLC 132.

Several manufacturers make PLCs 132 suitable for this application. The PLC 132 preferably receives data from and transmits data to operational components of the system (e.g. sensors 130, 142; valves 138, 140, 146; motor advance 135; boiler 136) at a sampling rate of one sample per five seconds or faster. The PLC 132 preferably also includes a communications port that is capable of communicating with a conventional personal computer 148. FIG. 3 shows the computer 148 communicating with the PLC 132 via dashed line 150. It should be understood that the computer 148 may communicate by any number of means with the PLC 132, such as over an internal network, over an internet connection, wirelessly, etc. In addition, while it may be desirable to have the computer 148 on-site at the pasteurization facility, the computer 148 or one or more additional computers 148 may be located remotely. Typically, the computer 148 will have a display and a user interface such as a keyboard and mouse or a touch screen whereas the PLC 132 might not have a display and user interface. The PLC 132 will typically be programmed via the communication link 150 between the computer 148 and the PLC 132. Desirably, the computer 148 receives data from the PLC 132 for each batch of shell eggs being pasteurized. Also, desirably real-time data from the temperature sensors 130, the status of the heating and/or cooling system, the flow rate of compressed air 146, and optionally the status of advance motor 135 are provided to the computer 148 in real-time. The real-time data can be viewed on the remote computer 148, and can also be stored for later use if necessary.

The following Example 1 describes experiments supporting to the discovery that the D-value for Salmonella Enteritidis in yolk at desirable pasteurization temperatures when the eggs are not refrigerated (or are suitably tempered at room temperature) is substantially less than the D-value when the eggs are refrigerated prior to pasteurization. Example 2 describes the collection of additional thermal validation data and development of a D-value pasteurization model, which provides D-values at typical pasteurization temperatures above 128° F. for shell eggs that are unrefrigerated (or are suitably tempered at room temperature) prior to pasteurization. The claimed invention is not limited by the specific information disclosed in these examples but is defined by the appended claims.

### Example 1

### Experimental Results for Salmonella Enteritidis at 133° F.

A thermal validation study was conducted to assess the thermal inactivation rate of a cocktail of Salmonella species including Salmonella Enteritidis in Medium and Large shell eggs when stored at a refrigerated temperature of 45° F., stored at 45° F. and then tempered at 65° F., or stored at 65° F. without refrigeration, and then pasteurized at 133° F.

Laboratory studies are generally accepted as alternatives to in-plant validations. Intact shell eggs were inoculated at high levels of Salmonella, and were heat treated in a circulating water bath set at 133° F. (±0.5° F.). After treatments, treated samples were analyzed for surviving Salmonella and the thermal inactivation rates were determined. Two production lots, representing two separate manufacturing dates, and 5-10 samples per lot at each time point were used for the validation study, in accordance with NACMCF guidelines, NACMCF Executive Secretariat, 2010, Parameters for determining inoculated pack/challenge study protocols. J. Food Prot. 73(1):140-202. Table 1 summarizes the test runs that were conducted.

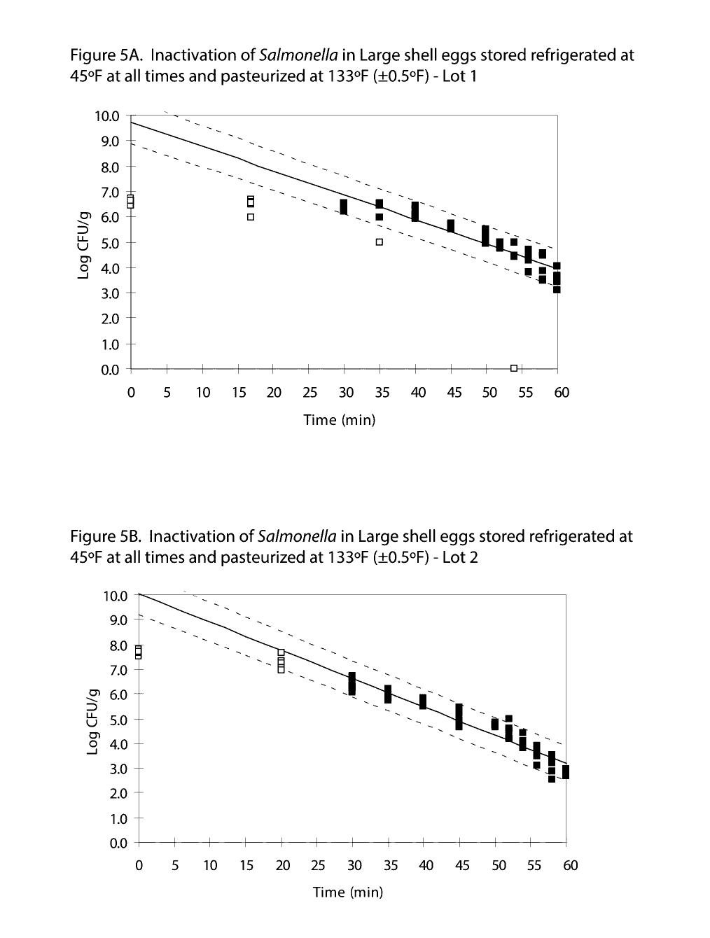

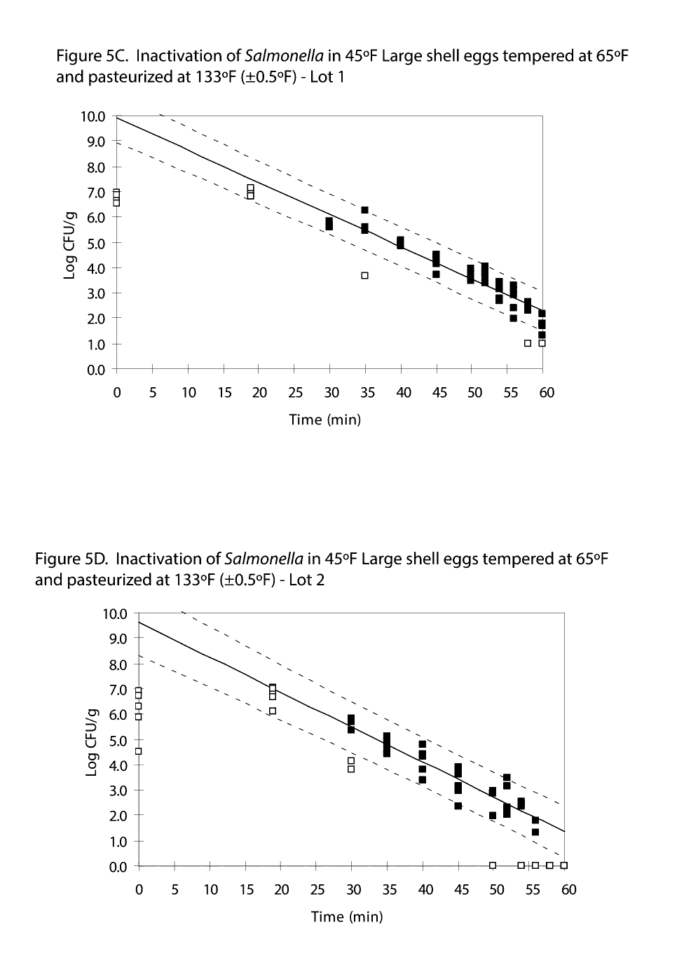

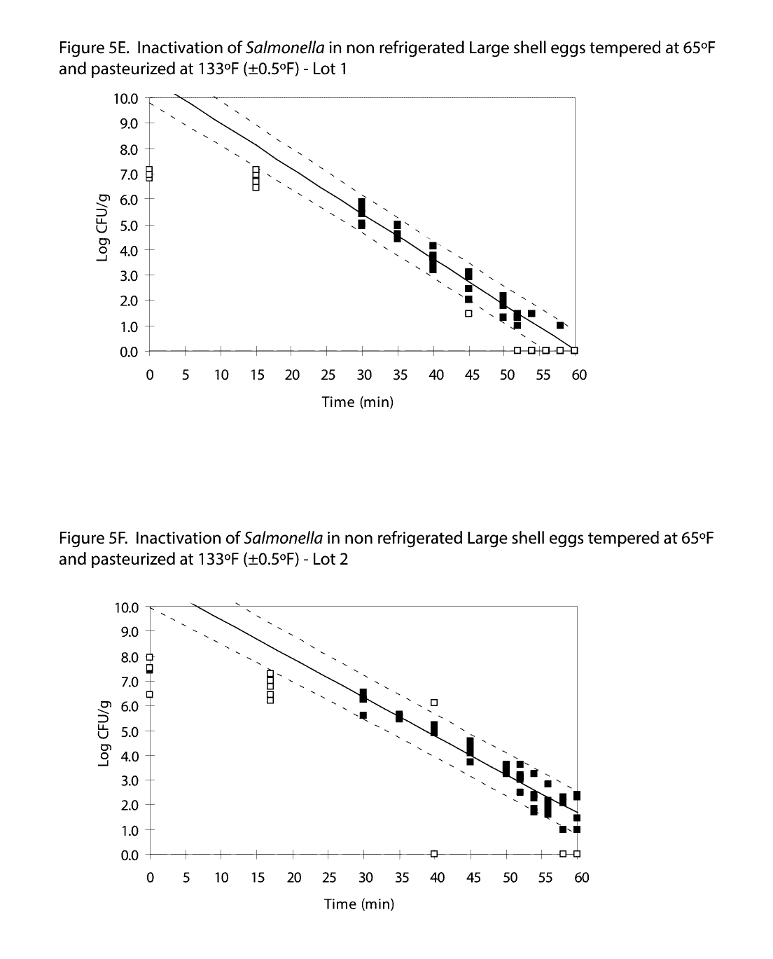

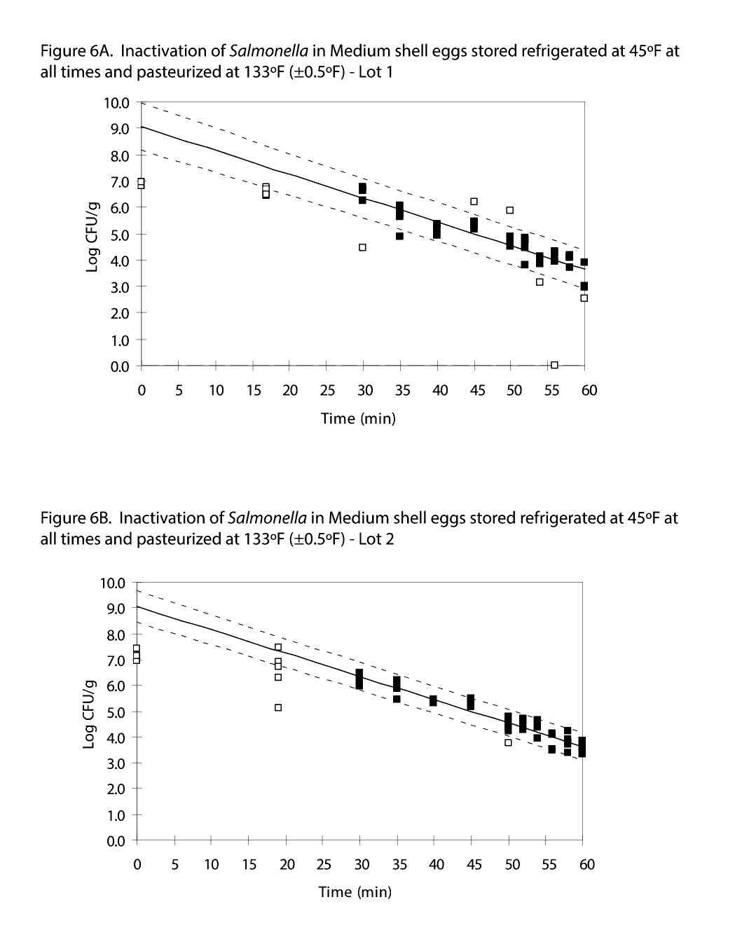

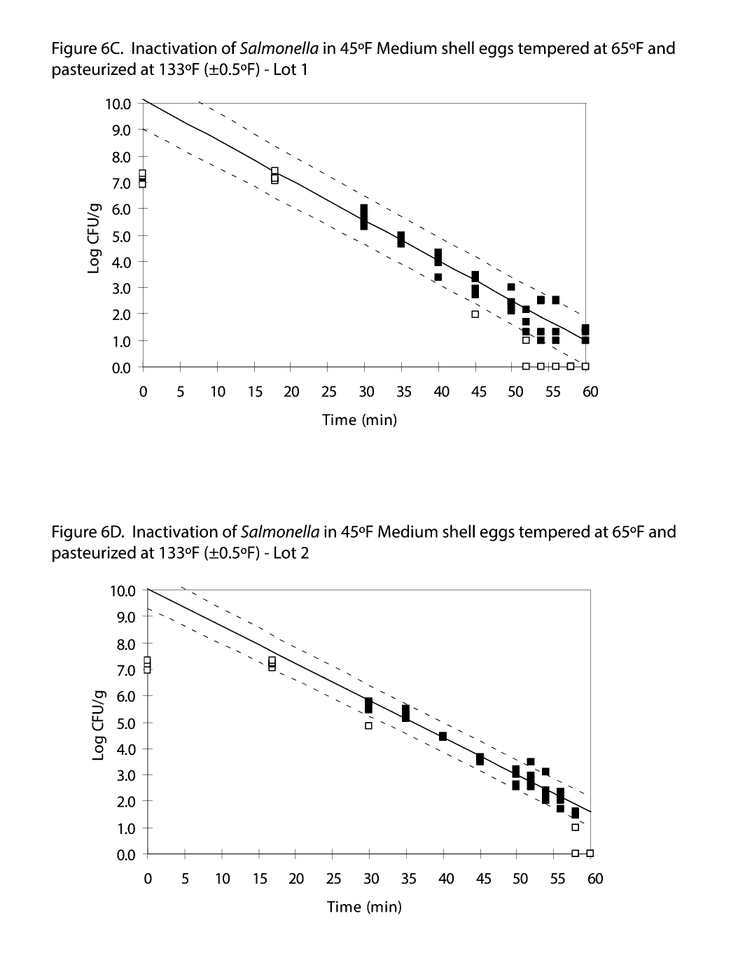

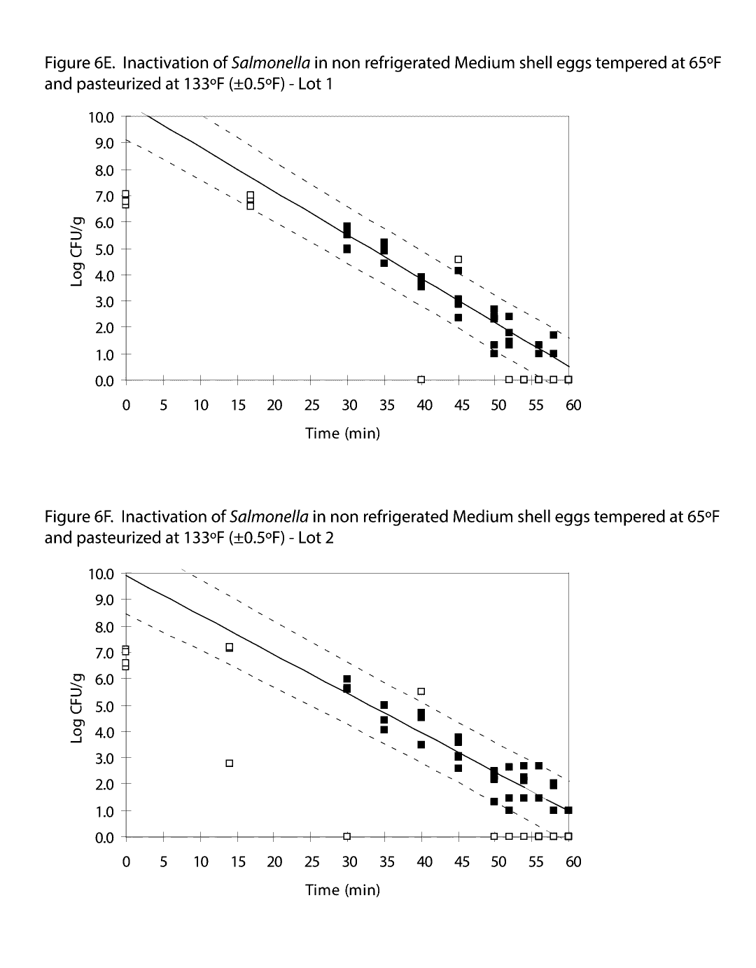

TABLE 1

| Egg size | Storage conditions | Trial |
|---|---|---|
| Large | stored refrigerated at 45° F. at all times | 1 |
| | (FIGS. 5A and 5B) | 2 |
| | 45° F. eggs tempered at 65° F. | 1 |
| | (FIGS. 5C and 5D) | 2 |
| | Non refrigerated eggs tempered at 65° F. | 1 |
| | (FIGS. 5E and 5F) | 2 |
| Medium | stored refrigerated at 45° F. at all times | 1 |
| | (FIGS. 6A and 6B) | 2 |
| | 45° F. eggs tempered at 65° F. | 1 |
| | (FIGS. 6C and 6D) | 2 |
| | Non refrigerated eggs tempered at 65° F. | 1 |
| | (FIGS. 6E and 6F) | 2 |

### Example 1

### Preparation of Salmonella and Inoculation

Samples of shell eggs were tempered at 45° F. or 65° F. for about 3 days until use.

The following six strains of Salmonella were used. These strains were used in previous thermal validation studies and have been maintained in the Silliker Inc., Food Science Center (FSC) culture collection (FSC-CC).

| Microorganisms | SLR Number |
|---|---|
| Salmonella Enteritidis | 267 |
| Salmonella Enteritidis | 268 |
| Salmonella Enteritidis | 269 |
| Salmonella Typhimurium | 449 |
| Salmonella Heidelberg | 539 |
| Salmonella Othmarschen | 544 |

The purity of each strain of Salmonella was verified by streak plating on xylose lysine desoxycholate (XLD) agar. The plates were incubated for 24 h at 35° C. Typical colonies were considered confirmatory. Strains of Salmonella were cultivated in tryptic soy broth (TSB) on two consecutive days and incubated at 35° C. for 24 h. The cultures were mixed to prepare a composite culture that contained approximately equal numbers of cells of each strain. The composite culture was centrifuged at 7,000 rpm for 15 min, the supernatant discarded and the bacterial pellet suspended in yolk.

Grade AA eggs were used in the study. A small hole was made in the shell of each egg where the air sac is located. A 2.5 in, 20-gauge syringe needle with a 1 cc syringe (BD, Franklin Lakes, N.J.) was used to inject 0.1 ml of composite culture into the yolk of each egg. The hole was sealed subsequently with a sealant (SEAL ALL, Eclectic Products, Pineville, La.) and set for 60 minutes at the pre-conditioning temperatures of 45° F. and 65° F. for bacterial attachment.

### Example 1

### Thermal Treatment

Inoculated eggs were placed in conventional flats and subjected to a thermal treatment at 133° F. (±0.5° F.) in a temperature controlled water bath. The water bath was pre-warmed to 133° F. Seventy inoculated eggs and 2 eggs with thermocouple to monitor the yolk temperature were placed in the water bath. Five eggs were removed from the water bath at timed intervals and analyzed for Salmonella. In addition, five eggs without thermal treatment were used to determine the inoculation level of eggs. Two trials were performed for each temperature using two separate lots. The temperature of the thermocouple prepared eggs and water bath were monitored using Yokagawa MV 1000 portable hybrid recorder (Shenandoah, Ga.) with T-type thermocouples. Five samples were pulled initially, initially (0 min), at 128° F. internal temperature and after 30 min, 35 min, 40 min, 45 min, 50 min, 52 min, 54 min, 56 min, 58 min, and 60 min exposure times.

### Example 1

### Microbiological Analysis

Thermally processed and control eggs were soaked in 25 ppm iodine for 1 minute followed by a soak for 5 min in 70% ethanol. The eggs were then blotted dry and cracked opened. The yolk was separated from the white and analyzed. The yolk was blended with 9 volumes of Butterfield's phosphate diluent in a Stomacher lab blender to form a 1:10 homogenate. Ten fold serial dilutions were prepared with the same diluent and plated using trypticase soy agar to enumerate the number of surviving Salmonella. The method of analysis is outlined in Table 2.

TABLE 2

| | | Incubation Time/ |
|---|---|---|
| Test | Medium | Temperature/Atmosphere |
| Salmonella | Typticase soy agar | 48 hours/35° C./aerobic |

### Example 1

### Results and Discussion

As mentioned, for each egg size, two experimental trials using two different lots were conducted. A total of five eggs were pulled at each time point. Non-heat-treated eggs were designated as 0 minutes. The base ten logarithms of the plate counts for Salmonella were plotted against pasteurization time and the best fit line was statistically determined by least squares linear regression. The D-value is the time required for the population to decrease by 90% or 1-log when held at a certain temperature. Mathematically, it is the negative inverse of the slope of the regression line. The D-value plots for Large eggs are shown in FIGS. 5A through 5F, and the D-value plots for Medium eggs are shown in FIGS. 6A through 6F. Solid squares are the plotted number of colony forming units per gram (CFU/g) expressed as a logarithm used for the determination of the D-value. Open squares are colony count data not used in the regression. A solid line is the slope of the least squares linear regression and the dashed lines describe the low and high 95% confidence intervals for the regression. For each run, a D-value was determined. The D-value results generated in the experiment of Example 1 represent the thermal lethality values at the internal yolk temperature of 133° F., and the effect of three storage conditions 1) refrigerated at 45° F. at all times, 2) refrigerated and then tempered at 65° F., and 3) non-refrigerated and tempered at 65° F., on the calculated D-values. Table 3 lists calculated D-values for Salmonella in Medium and Large shell eggs refrigerated at 45° F. and pasteurized at 133° F. (±0.5° F.) compared to refrigerated and unrefrigerated eggs tempered to 65° F. and pasteurized at 133° F. (±0.5° F.).

TABLE 3

| Tempered temperature | Trial | Large Egg | Medium Egg |
|---|---|---|---|
| Stored refrigerated at 45° F. at all times | 1 | 10.39 min, r² = 0.87 | 11.08 min, r² = 0.86 |
| | 2 | 7.87 min, r² = 0.91 | 11.05 min, r² = 0.92 |
| 45° F. eggs tempered at 65° F. | 1 | 5.59 min, r² = 0.95 | 6.02 min, r² = 0.91 |
| | 2 | 8.75 min, r² = 0.91 | 6.56 min, r² = 0.92 |
| Non refrigerated eggs tempered at 65° F. | 1 | 7.26 min, r² = 0.86 | 7.10 min, r² = 0.95 |
| | 2 | 6.42 min ,r² = 0.93 | 6.72 min, r² = 0.86 |

The calculated D-values for the yolk at 133° F. for unrefrigerated shell eggs tempered at 65° F. and for refrigerated shell eggs tempered at 65° F. for about 3 days were considerable smaller compared to those for eggs stored and refrigerated at 45° F. at all times prior to pasteurization. The findings of this study are in accordance with the previous studies where the model predicted the D-values for yolk at 133° F. in the range of 8.01 min to 10.81 min for 3-5 days old Large size shell eggs stored at 41° F., see also the incorporated Davidson '538 patent which discloses a 5 D-value plot based the previous study, as well as Schuman et al. referenced above. The reduction of Salmonella in the eggs tempered at 65° F. may be due to different physicochemical interactions in the yolk.

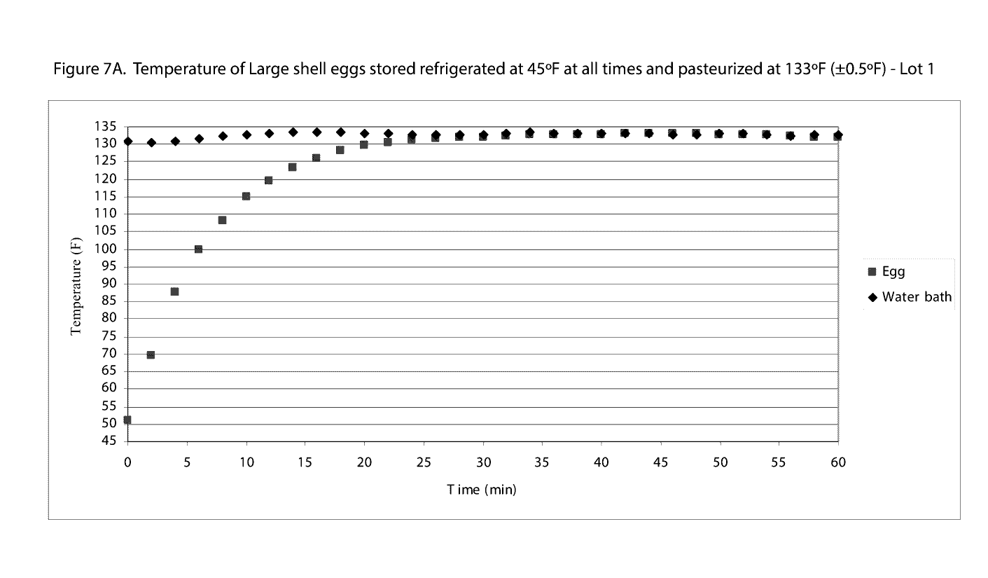

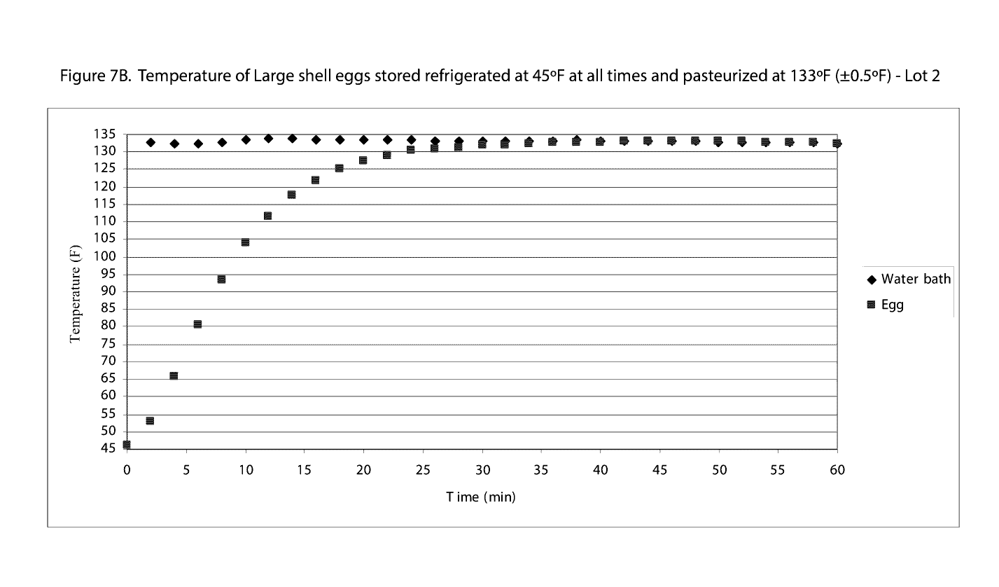

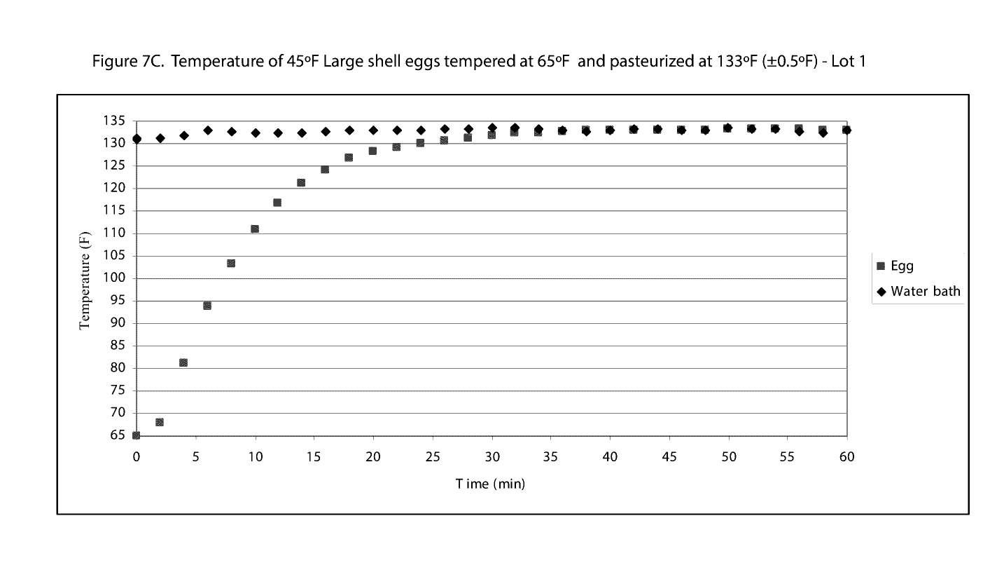

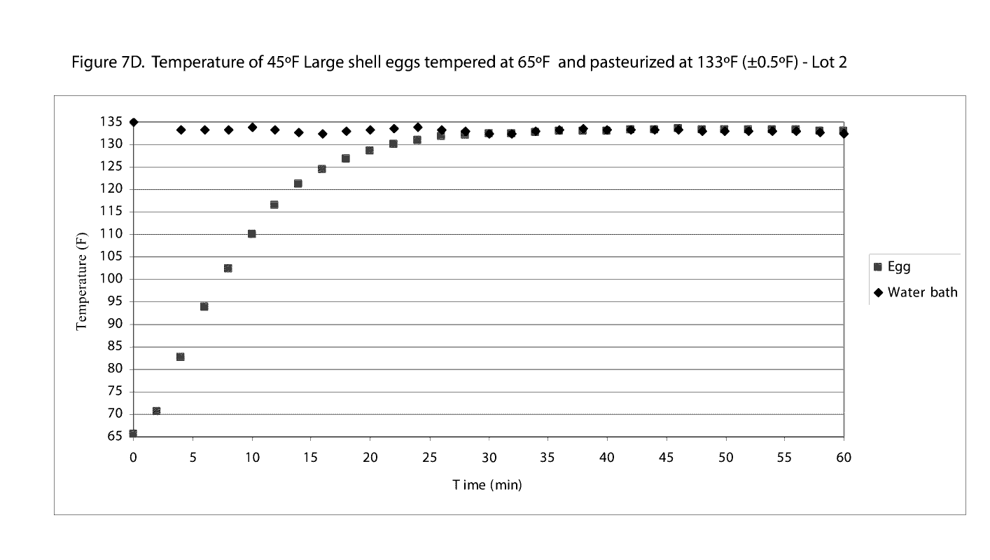

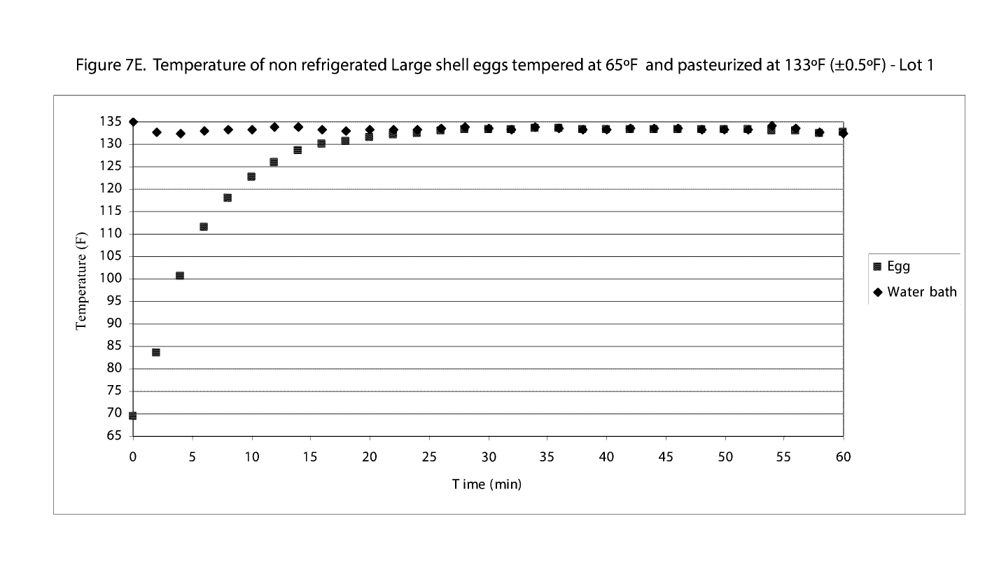

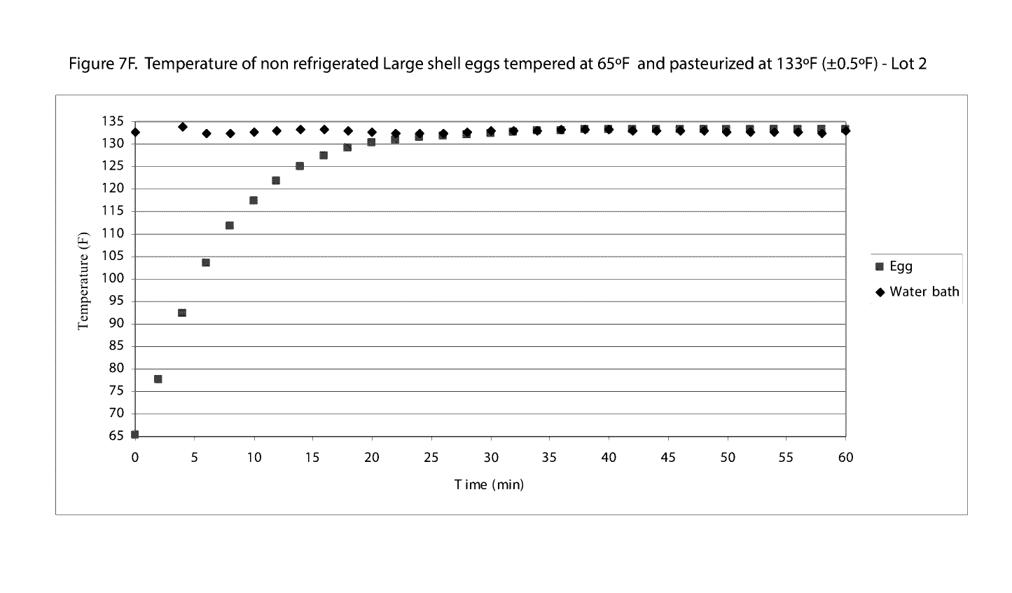

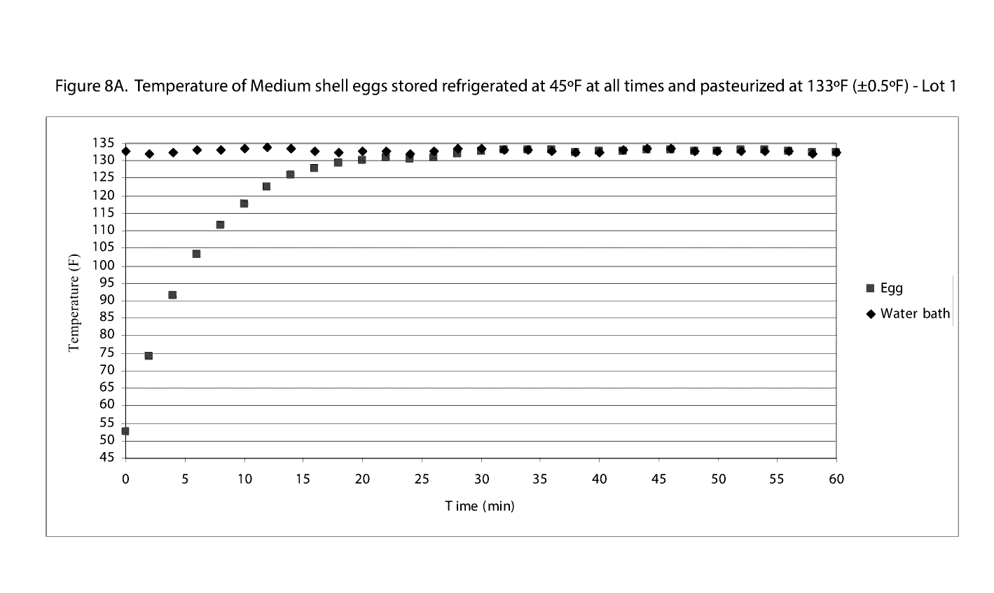

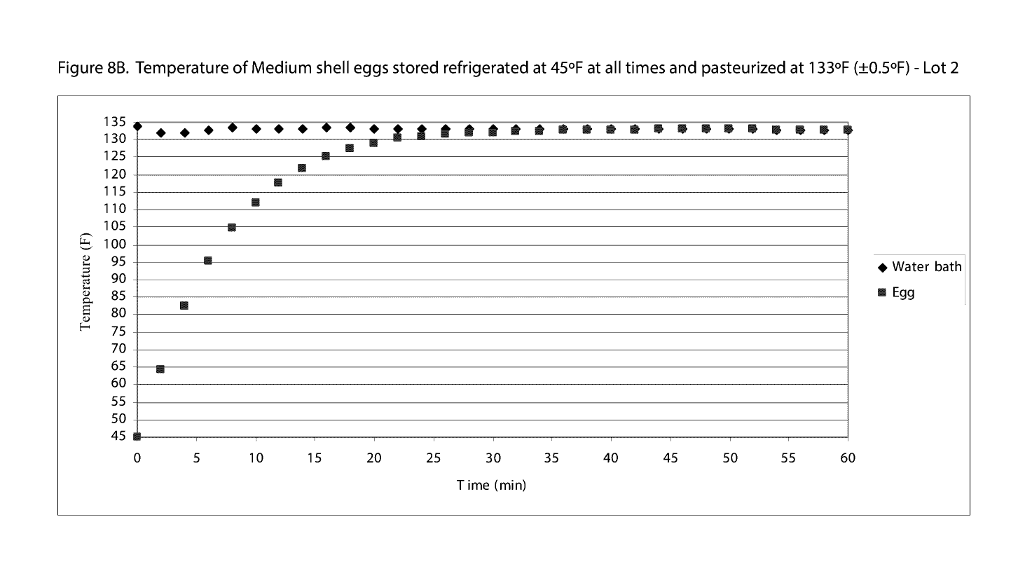

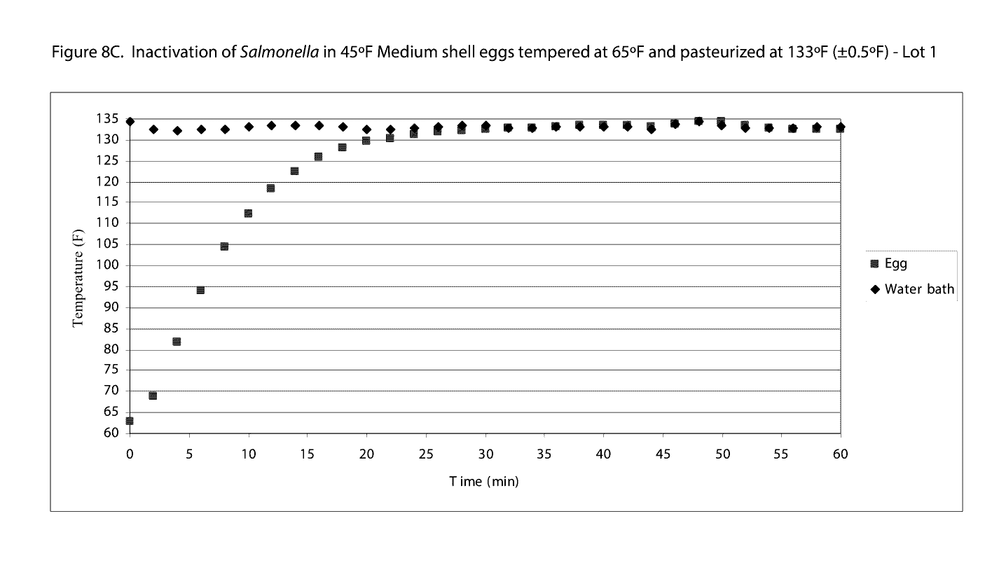

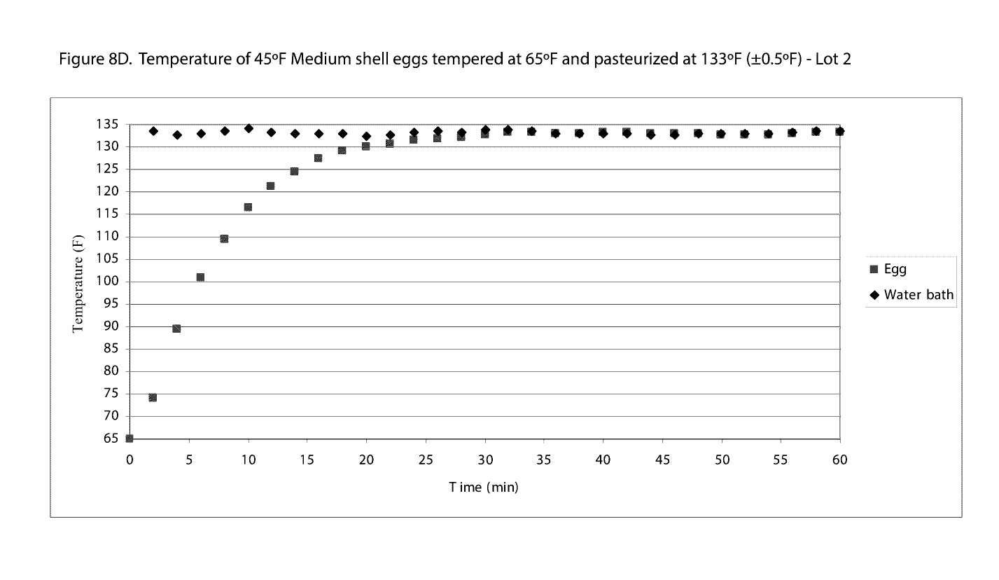

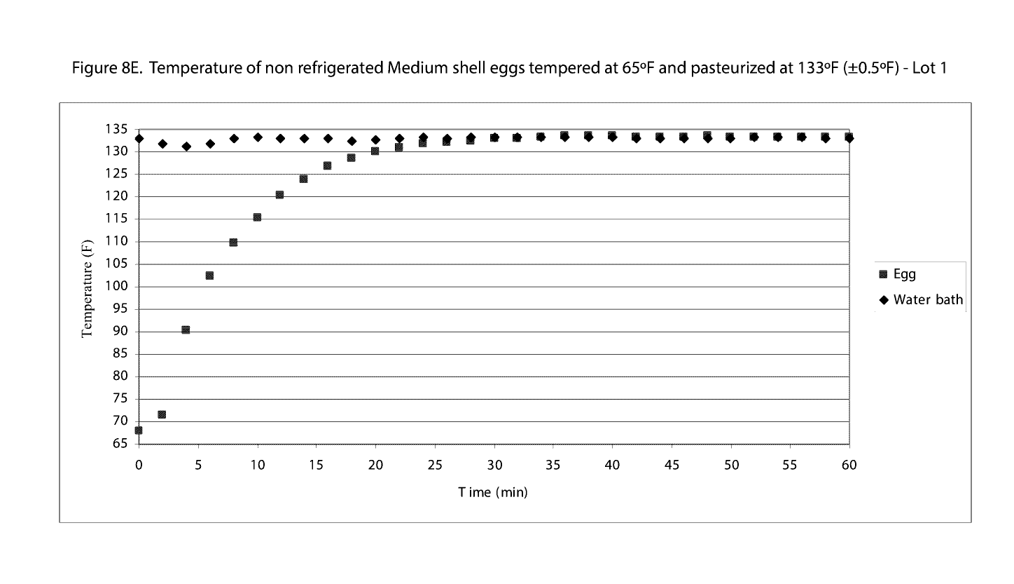

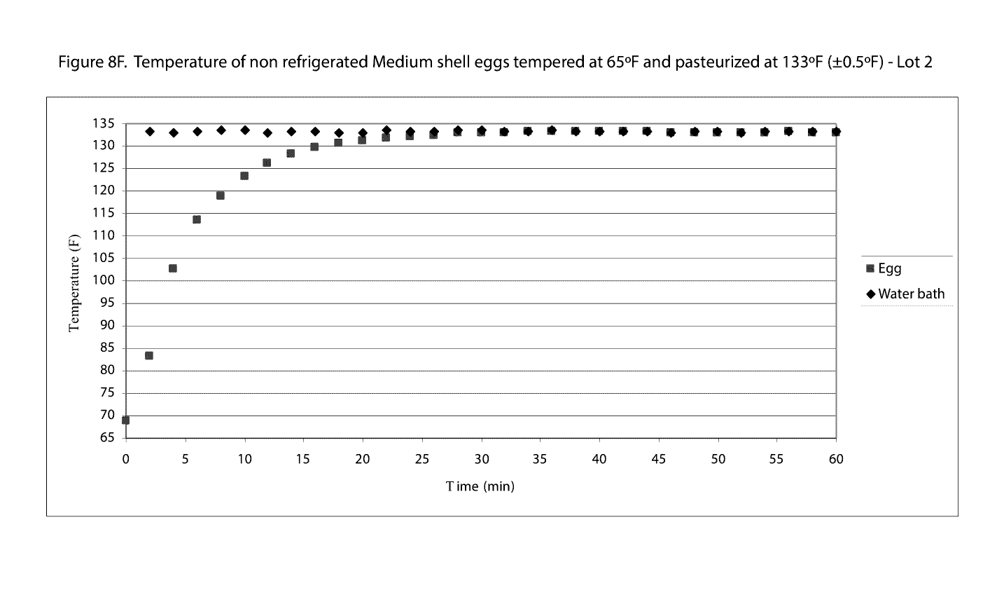

The temperature of the yolk and water bath was monitored during the pasteurization trials. Data for Large eggs trials is in FIGS. 7A through 7F and data for Medium eggs trials is in FIGS. 7A through 7F.

The lower D-value means that pasteurization of the in-shell chicken eggs in a water bath at 133° F. can achieve the required 5-logkill if the eggs are unrefrigerated or tempered adequately to room temperature prior to pasteurization in less time than if the eggs are refrigerated. The D-value for unrefrigerated eggs and eggs adequately tempered to room temperature are statistically equivalent.

### Example 2

### Experimental Results for D-Value Pasteurization Model

With the discovery from Example 1 that calculated D-values for Salmonella in the yolk of Medium and Large shell eggs refrigerated at 45° F. and pasteurized at 133° F. was higher than the calculated D-values for Salmonella in the yolk of Medium and Large shell eggs not refrigerated, or adequately tempered to room temperature, prior to pasteurization at 133° F., an experiment was conducted to determine a D-value pasteurization model for shell eggs that are not refrigerated, or are adequately tempered to room temperature, prior to pasteurization. To accomplish this task, a thermal validation study was conducted to assess the thermal inactivation D-values and the z-value of Salmonella in unrefrigerated Large shell eggs when pasteurized at 131° F. to 135° F., with the D-values for Salmonella at 133° F. being determined previously in Example 1.

Intact unrefrigerated Large shell eggs that were three to five days old were used. The shell eggs were tempered at 65° F. for about 3 days until use. The unrefrigerated Large shell eggs were inoculated at high levels of Salmonella in the same manner as described above in Example 1, and heat treated in a circulating water bath set at 131° F., 134° F. and 135° F. After treatments, treated samples were analyzed for surviving Salmonella and the thermal inactivation rates were determined. As in Example 1, the following six strains of Salmonella were used. These strains as mentioned are maintained in the Silliker Inc., Food Science Center (FSC) culture collection (FSC-CC).

| Microorganisms | SLR Number |
|---|---|
| Salmonella Enteritidis | 267 |
| Salmonella Enteritidis | 268 |
| Salmonella Enteritidis | 269 |
| Salmonella Typhimurium | 449 |
| Salmonella Heidelberg | 539 |
| Salmonella Othmarschen | 544 |

Similar to Example 1, the purity of the Salmonella stock cultures was verified by streak plating on xylose lysine desoxycholate (XLD) agar plates. The plates were incubated at 35° C. for 24 h. In addition, an isolated colony of each strain from the stock culture was confirmed by serological tests. Strains of Salmonella were cultivated in tryptic soy broth (TSB) on two consecutive days and incubated at 35° C. for 24 h. The cultures were mixed to prepare a composite culture that contained approximately equal numbers of cells of each strain. The composite culture was centrifuged at 7,000 rpm for 15 min, the supernatant discarded and the bacterial pellet suspended in yolk.

Similar to Example 1, a small hole was made in the shell of each egg where the air sac is located. A 2.5 in, 20-gauge syringe needle with a 1 cc syringe (BD, Franklin Lakes, N.J.) was used to inject 0.1 ml of composite culture into the yolk of each egg. The hole was sealed subsequently with a sealant (SEAL ALL, Eclectic Products, Pineville, La.) and set for 60 minutes at 65° F. for bacterial attachment.

### Example 2

### Thermal Treatment

Again like Example 1, inoculated eggs were placed in conventional flats and subjected to a thermal treatment in a temperature controlled water bath. The water bath was pre-warmed to the test temperature. Inoculated eggs and two eggs with thermocouple to monitor the yolk temperature were placed in the water bath. Five eggs were removed from the water bath at timed intervals and analyzed for Salmonella. In addition, five eggs without thermal treatment were used to determine the inoculation level of eggs. The temperature of the thermocouple prepared eggs and water bath were monitored using Yokagawa MV 1000 portable hybrid recorder (Shenandoah, Ga.) with T-type thermocouples.

### Example 2

### Microbiological Analysis

As discussed in Example 1, thermally processed and control eggs were soaked in 25 ppm iodine for 1 minute followed by a soak for 5 min in 70% ethanol. The eggs were then blotted dry and cracked opened. The yolk was separated from the white and analyzed. The yolk was blended with nine (9) volumes of Butterfield's phosphate diluent in a Stomacher lab blender to form a 1:10 homogenate. Ten fold serial dilutions were prepared with the same diluent and plated using trypticase soy agar to enumerate the number of surviving Salmonella. The method of microbiological analysis is outlined in Table 2 above referenced in Example 1.

### Example 2

### Results and Discussion

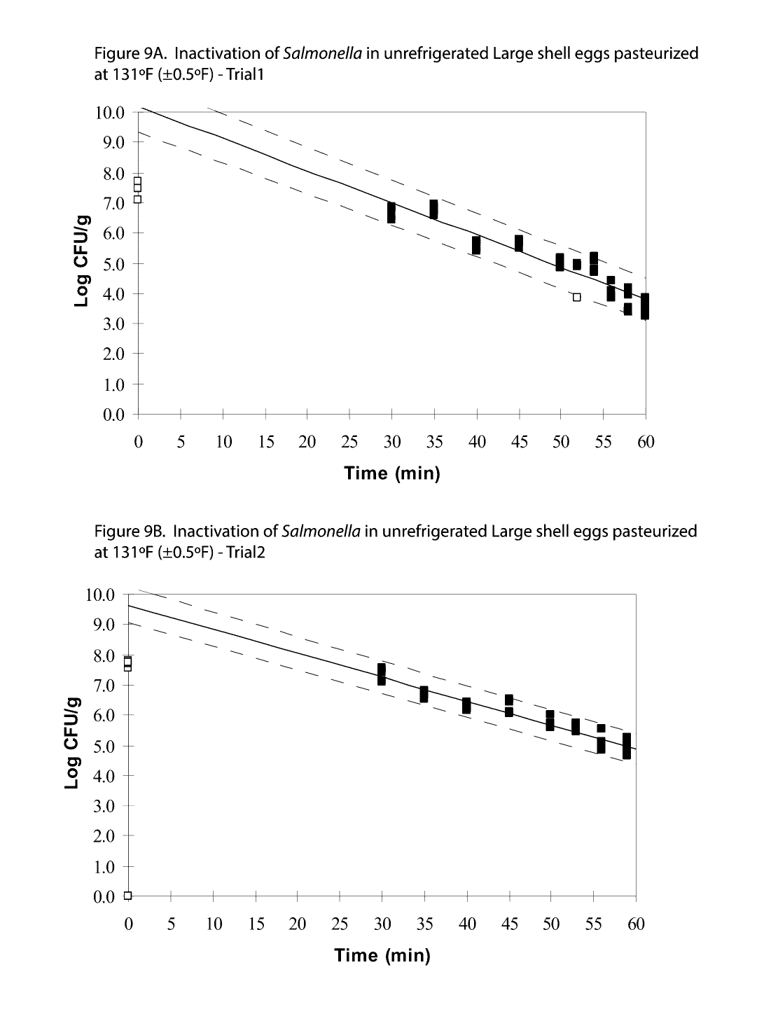

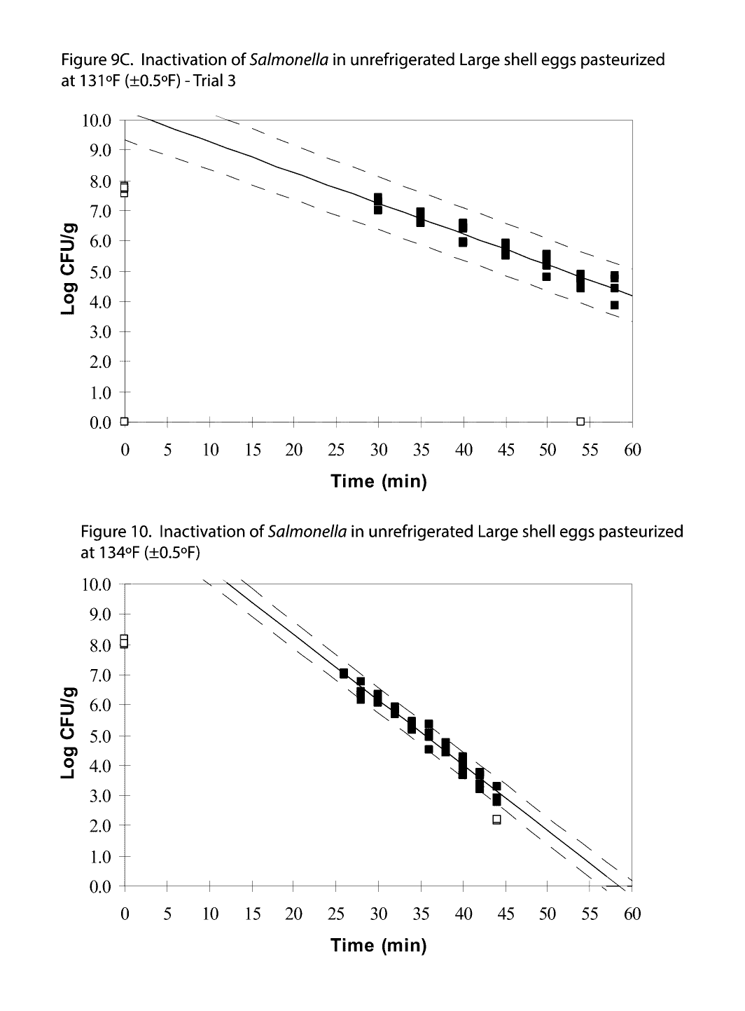

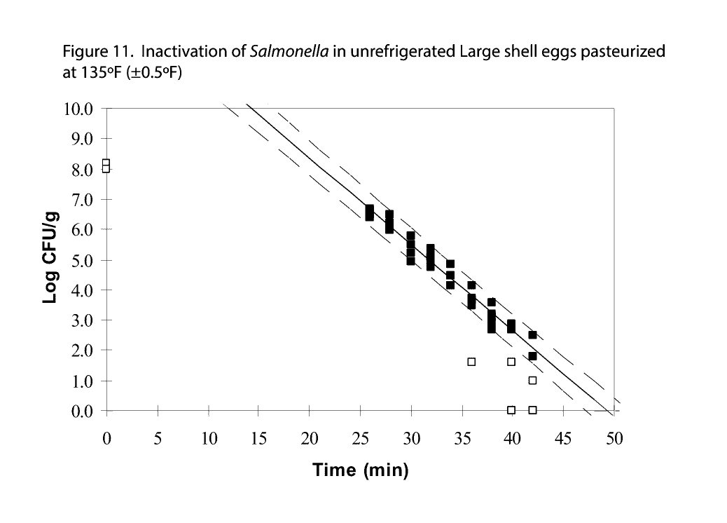

A total of five eggs were pulled at each time point. Non-heated treated eggs were designated as 0 minutes. The 128° F. value eggs were excluded from this determination due to minimal reduction during heating. The base ten logarithms of the plate counts for Salmonella were plotted against pasteurization time and the best fit line was statistically determined by least squares linear regression. As mentioned, the D-value is the time required for the population to decrease by 90% or 1-log when held at a certain temperature. Mathematically, it is the negative inverse of the slope of the regression line. The D-value plots for the 131° F. water baths are plotted in FIGS. 9A, 9B, and 9C. The D-value plot for the 134° F. water bath is plotted in FIG. 10. The D-value plot for the 135° F. water bath is plotted in FIG. 11. Three trials were performed for 131° F., one trial for 134° F. and 135° F. because of the low r-squared value, see Table 4. In FIGS. 9A, 9B, 9C, 10 and 11, solid squares are the plotted number of colony forming units per gram (CFU/g) expressed as a logarithm used for the determination of the D-value. Open squares are colony count data not used in the regression. A solid line is the slope of the least squares linear regression and the dashed lines describe the low and high 95% confidence intervals for the regression. A D-value was determined for each run. The D-value results generated represent the thermal lethality values at the internal yolk temperature of 131° F., 134° F. and 135° F. (Table 4), for shell eggs having a start temperature of 65° F. The D-value for 133° F. for a Large shell egg having a start temperature of 65° F. was previously determined in Example 1, FIGS. 5C-5F, Table 3.

Table 4. D-values of Salmonella in unrefrigerated large shell eggs when tempered at 65° F. and pasteurized at 131° F., 134° F. and 135° F. (±0.5° F.).

TABLE 4

| | | |
|---|---|---|
| Temperature | Trial | D- value (r²) |

| | | | |
|---|---|---|---|
| 131° F. | 1 | 9.40 min | (r² = 0.90) |
| | 2 | 12.54 min | (r² = 0.92) |
| | 3 | 9.81 min | (r² = 0.91) |
| 134° F. | 1 | 4.63 min | (r² = 0.97) |
| 135° F. | 1 | 3.50 min | (r2 = 0.97) |

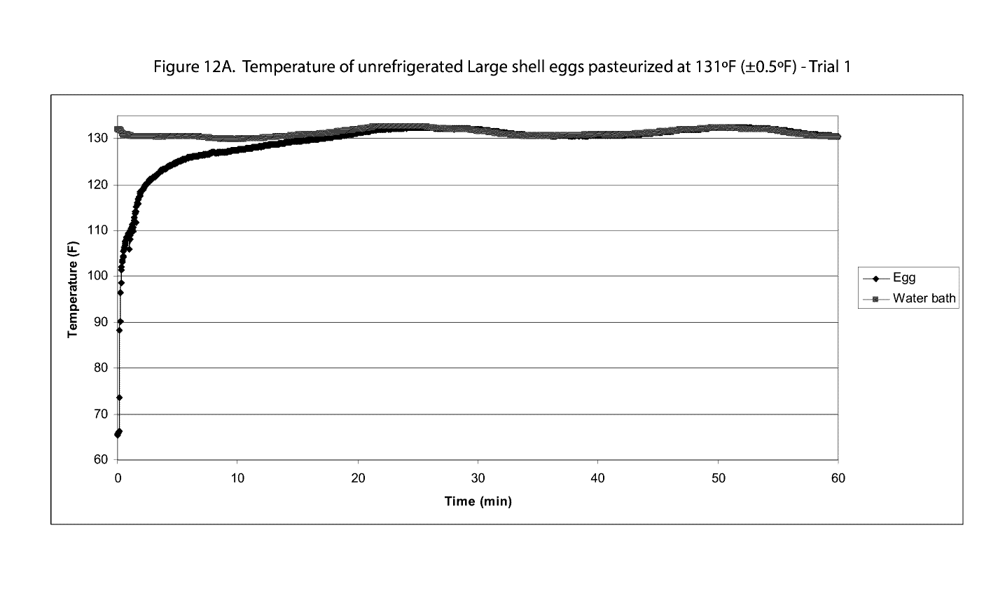

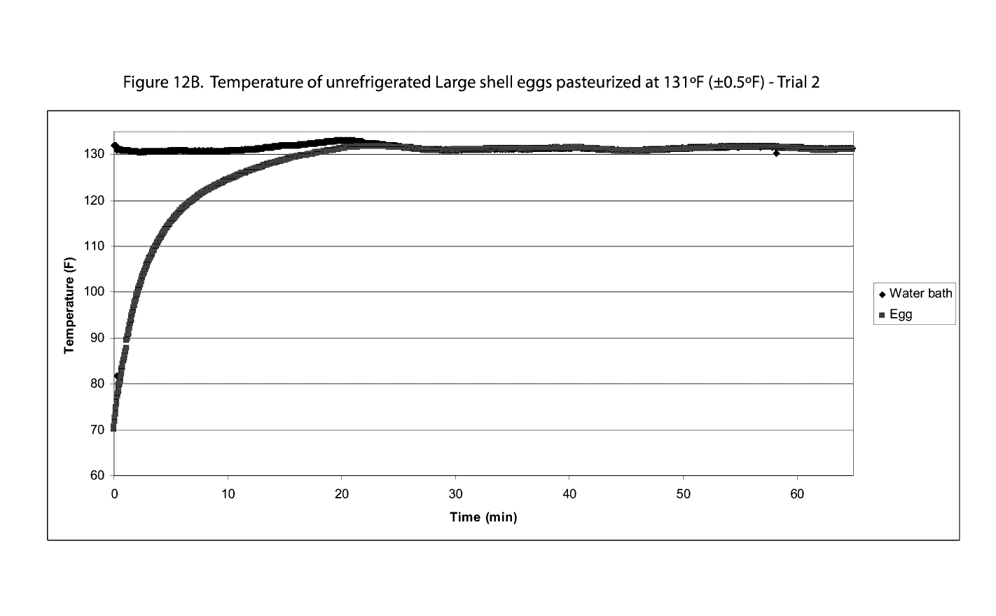

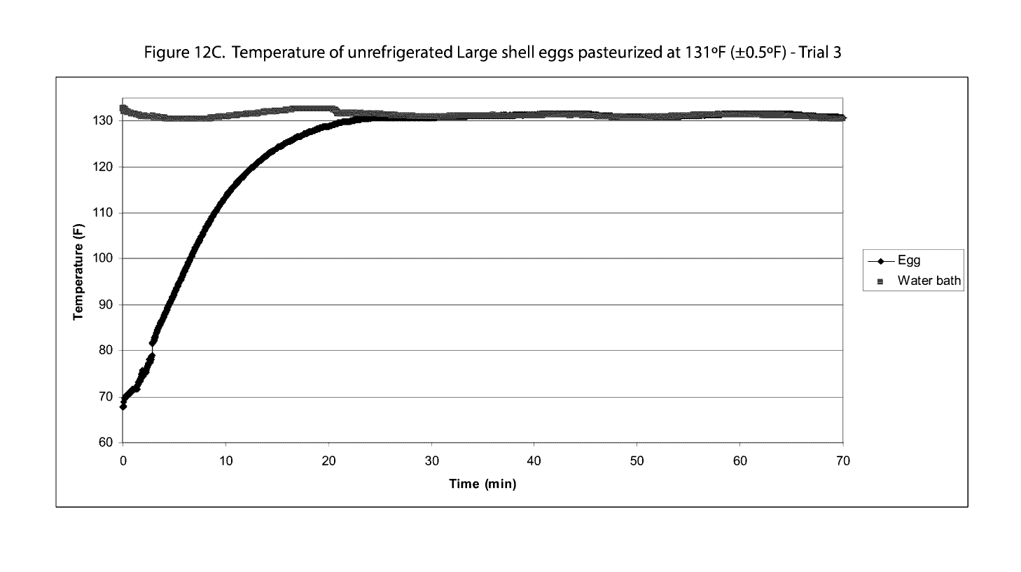

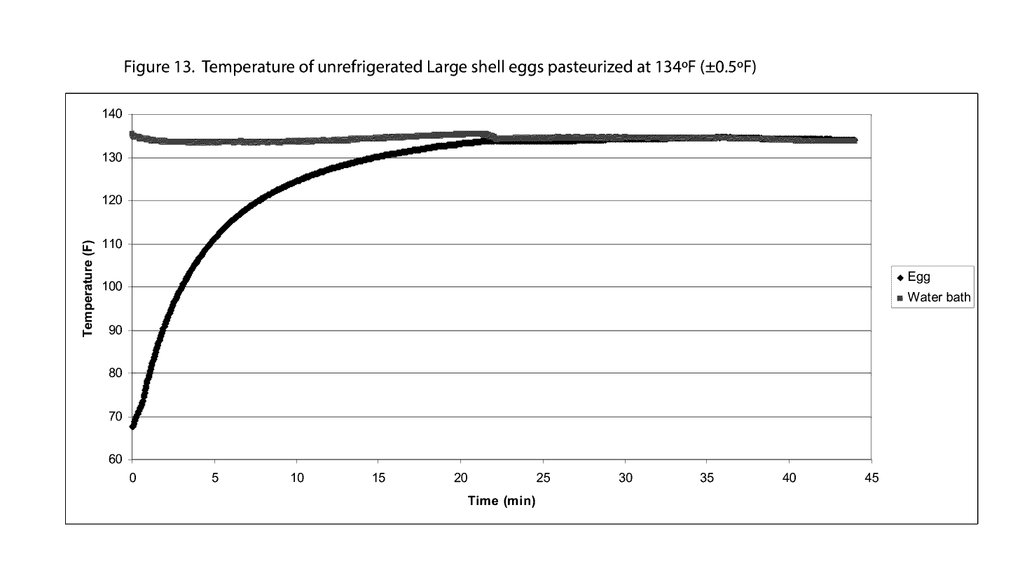

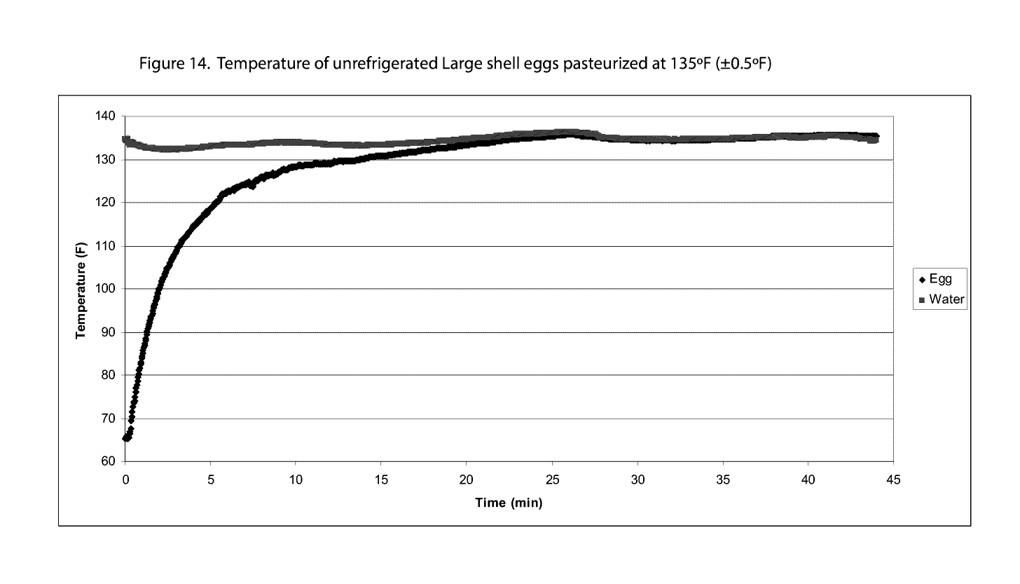

The temperature of the yolk and water bath was also monitored during pasteurization trials. The temperatures for the three D-value test in a 131° F. water bath are shown in FIGS. 12A, 12B and 12C. The temperatures for the D-value test in the 134° F. water bath are shown in FIG. 13. The temperatures for the D-value test in the 135° F. water bath are shown in FIG. 14.

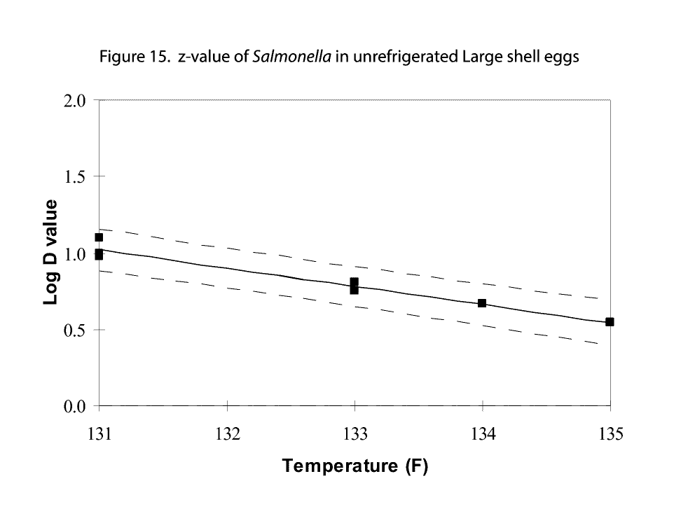

The z-value represents the number of ° F. required for the D-value to proceed through one log cycle. Mathematically, it is the negative inverse slope of the regression line of the logarithm of D-values and temperature. The regression line is graphically shown in FIG. 15, and the experimental z-value is 8.38° F. D-values calculated from the z-value determination are understood in the art to be better estimates of the true D-values for a temperature than simple experimental D-values found for each temperature. The experimental z-value is 8.38° F. for this experiment. Solid squares are the plotted number of D-values expressed as a logarithm used for the determination of the z-value. A solid line is the slope of the least squares linear regression and the dashed lines describe the low and high 95% confidence intervals for the regression. The z-value is the negative inverse of the slope of the regression line. The calculated D-values for unrefrigerated shell eggs are presented in Table 5.

TABLE 5

| Pasteurization temperature | D-value | 95% Confidence Interval, D-value |
|---|---|---|
| (° F.) | (min) | (min) |
| 130 | 13.78 | 9.74-19.50 |
| 131 | 10.47 | 7.63-14.37 |
| 132 | 7.96 | 5.9-10.73 |
| 133 | 6.04 | 4.49-8.14 |
| 134 | 4.59 | 3.36-6.28 |
| 135 | 3.49 | 2.48-4.91 |
| 136 | 2.65 | 1.81-3.88 |

The following Equation (1) provides a empirically verified D-value pasteurization model for calculating D-values of Salmonella in the yolk of an unrefrigerated Large shell for a given yolk pasteurization temperature:

D-value=10̂(−0.11931×Temperature+16.64978) (1)

The D-value pasteurization model in Equation (1) is used to determine total required pasteurization time at a given water bath temperature by using yolk temperature come up time data and accumulating log kill, e.g. on a minute by minute basis, after the yolk temperature increases above 128° F. until the required time for a 5-logreduction is achieved.

For example, using a tempered start temperature of 65° F., the required time to achieve a 5-log reduction of Salmonella Enteritidis for unrefrigerated Large shell eggs in a 133° F. water bath like that shown in FIG. 1, using the temperature data in FIGS. 7E and 7F, is conservatively calculated at 50 minutes. As another example, using a tempered start temperature of 65° F., the required time to achieve a 5-log reduction of Salmonella Enteritidis for unrefrigerated Large shell eggs in a 134° F. water bath like that shown in FIG. 1, using the temperature data in FIG. 13, is conservatively calculated at 45 minutes. In these calculations, the total dwell time is calculated with the chicken shell eggs not being removed from the water bath until the eggs have been in the water bath for an amount of time sufficient to achieve a statistical 5-log reduction of Salmonella Enteritidis that may have been present in the yolk according to the D-value pasteurization model of Equation (1). Of course, pasteurization occurs after the egg is removed from that heated water bath until the yolk cools below 128° F. Including log reduction of Salmonella Enteritidis after the Large eggs are removed from the heated water bath but before the yolk cools to below 128° F. will reduce the required dwell time from the 50 minute (133° F.) and 45 minute (134° F.) values calculated above.

The findings of this study were compared to the previous study, which is the basis for the Davidson '538 patent, where the model predicted the D-values for 3-5 days old Large size shell eggs stored at 41° F. The calculated D-values of the unrefrigerated Large shell eggs tempered at 65° F. were considerably smaller compared to the D-values previously calculated for refrigerated eggs.

The D-value pasteurization model calculated for unrefrigerated chicken eggs can be used to calculate required pasteurization times in pasteurization systems other than the type shown in FIG. 1. For example, the system in FIG. 1 provides uniform heating and temperature rise of the eggs throughout the stacks being pasteurized so it is appropriate to accumulate log kill during the time the yolk temperature increases from 128° F. to the water bath temperature. Equation (1) can be used, however, even in systems where it is not appropriate to accumulate log kill during come up time because yolk temperature rise is not uniform. Also, Equation (1) can be used to determine dwell times in systems having multiple heated water baths by applying yolk temperature data to determine the necessary dwell time in the various water baths. Further, while the calculated dwell time is predetermined in the exemplary embodiments, it is possible to calculate accumulated kill level per the D-value pasteurization model of Equation (1) while the system is operating to pasteurize as disclosed in U.S. Pat. No. 5,993,886, entitled “Method and Control System for Controlling Pasteurization of In-Shell Eggs,” by Louis Polster, issuing on Nov. 30, 1999, and hereby incorporated by reference.

Those skilled in the art will recognize that the D-value pasteurization model in Equation (1) is exemplary, and that further thermal validation studies on unrefrigerated eggs or eggs suitably tempered at a room temperature, e.g. in the range of about 60° F. to 85° F., may not result in an identical D-value model but are considered to be within the scope of the claimed invention. In addition, while the heated fluid pasteurization medium takes the form of a heated water bath in the exemplary embodiments, the heated fluid pasteurization medium can take other forms, such as heated humid air, or convection of heated air, or a combination of these heating techniques or others. The fundamental discovery of the inventors being that D-values for Salmonella Enteritidis in yolk at a given yolk temperature are substantially lower for shell eggs that are not refrigerated prior to pasteurization, or that are tempered at room temperature for a sufficient amount of time prior to pasteurization.

While the FDA presently requires a 5-log reduction of Salmonella Enteritidis in order for a chicken shell egg to be considered pasteurized, the use of a D-value pasteurization model calculated for unrefrigerated eggs is applicable to pasteurization to other log reduction levels.

### Surface Contamination

Thermal inactivation of Salmonella on the outer surface of shell eggs was also tested using the same strains listed above, namely Salmonella Enteritidis (267, 268, 269), Salmonella Typhimurium (449), Salmonella Heidelberg (539), and Salmonella Othmarschen (544). The minimum log reduction was calculated by subtracting the highest survivor log of the count from the treated samples from the lowest log of the count from the untreated inoculated samples. The data showed that in a pasteurization water bath at 133° F. Large eggs exceeded a minimum of 5-log reduction for surface contamination with a 10 minute exposure time. It is therefore clear that water bath pasteurization whether at 133° F. or 134° F. for around 50 minutes well exceeds the necessary time to eliminate Salmonella risk on the surface of the shell egg.

In the foregoing description, certain terms have been used for brevity, clarity, and understanding. No unnecessary limitations are to be inferred therefrom beyond the requirement of the prior art because such terms are used for descriptive purposes and are intended to be broadly construed. It is to be expected that various equivalents, alternatives and modifications are possible within the scope of the appended claims. Each limitation in the appended claims is intended to invoke interpretation under 35 U.S.C. §112, sixth paragraph, only if the terms “means for” or “step for” are explicitly recited in the respective limitation.

What is claimed is:

1. A method of pasteurizing a chicken shell egg comprising the steps of:
a. determining an empirically verified D-value pasteurization model for the statistical reduction of Salmonella Enteritidis in the yolk of chicken shell eggs, wherein the empirically verified D-value pasteurization model is determined by collecting thermal validation data on kill levels of Salmonella Enteritidis in chicken shell eggs that are inoculated, held at an unrefrigerated temperature prior to pasteurization and subsequently heated to suitable pasteurization temperatures;
b. heating a pasteurization medium to one or more desired pasteurization temperatures;
c. tempering the chicken egg to room temperature;
d. placing the tempered chicken egg in the heated pasteurization medium;
e. holding the chicken egg in the water bath for a time sufficient to ensure that both the yolk and albumen of the egg are pasteurized to achieve at least a statistical 5-log reduction of Salmonella Enteritidis that may have been present in the yolk in its unpasteurized form according to said D-value pasteurization model; and
f. removing the chicken egg from the pasteurization medium.

2. The method of claim 1 wherein the chicken egg is tempered to about 65° F. prior to being placed in the heated water bath.

3. The method of claim 1 wherein the tempered chicken egg is not refrigerated after it is laid and prior to tempering and placement into the heated pasteurization medium.

4. The method of claim 1 wherein the unrefrigerated chicken egg is a Large-sized egg, the heated pasteurization medium is water, the water bath is heated to 133° F.+/−0.5° F. while the egg is located within the water bath, and the time the chicken egg is held in the water bath is predetermined and no more than 50 minutes.

5. The method of claim 1 wherein the unrefrigerated chicken egg is a Large-sized egg, the heated pasteurization medium is water, the water bath is heated to 134° F.+/−0.5° F. while the egg is located within the water bath, and the time the chicken egg is held in the water bath is predetermined and no more than 45 minutes.

6. The method of claim 1 wherein the chicken egg is not removed from the pasteurization medium until it has been in the pasteurization medium for an amount of time sufficient to achieve a statistical 5-log reduction of Salmonella Enteritidis that may have been present in the yolk in its unpasteurized form according to said D-value pasteurization model, while the egg is held in the heated pasteurization medium.

7. The method of claim 1 wherein the predetermined time in the step e) is calculated to enable removal of the chicken egg from the heated pasteurization medium prior to achieving a statistical 5-log reduction of Salmonella Enteritidis that may have been present in the yolk in its unpasteurized form according to said D-value pasteurization model, wherein pasteurization continues after the egg is removed from that heated pasteurization medium until the yolk cools below 128° F. and the predetermined time in the pasteurization medium is sufficient to achieve said 5-log reduction before the yolk cools to below 128° F.

8. The method of claim 1 wherein the unrefrigerated temperature prior to the pasteurization when collecting thermal validation data to determine the empirically verified D-value pasteurization model for the statistical reduction of Salmonella Enteritidis in the yolk of chicken shell eggs is substantially about 65° F.

9. The method of claim 1 wherein the determination of the empirically verified D-value pasteurization model comprises inoculating chicken shell eggs with a Salmonella cocktail including at least one strains of Salmonella Enteritidis.

10. The method of claim 1 wherein the determination of the empirically verified D-value pasteurization model comprises inoculating chicken shell eggs with a Salmonella cocktail including strains of: Salmonella Enteritidis, Salmonella Typhimurium, Salmonella Heidelberg, and Salmonella Othmarschen.

11. The method of claim 1 wherein the pasteurization medium is a water bath heated with a heating system to achieve a temperature set point commensurate with the desired pasteurization temperature and is cooled with a cooling system when the temperature exceeds the temperature set point and approaches an upper temperature limit of not more than about 0.5° F. to 1.0° F. above the temperature set point.

12. The method of claim 1 wherein heated pasteurization medium is a water bath and the step of placing the egg in the water bath comprises enveloping at least one stack of a plurality of layers of eggs of a particular size in the water bath, said stack comprising layers of egg filled flats with at least thirty eggs in each flat.

13. The method of claim 7 further comprising supplying air bubbles into the heated water bath to perturbate the water across the surface of the eggs in the stack of eggs.

14. A shell egg pasteurization system comprising:
a pasteurization bath containing liquid water and having a series of continuous, in-line stage locations for batches of stacked chicken shell eggs;
multiple temperature sensors in the bath for measuring the water temperature in the bath;
a heating system that operates to increase the temperature of the water in each zone of the bath to a temperature set point value, said heating system including multiple independently controlled heating elements in the bath;
an air bubble supply system that injects air bubbles into the bath underneath the heating elements in order to agitate water in the vicinity of the heating elements and stacked chicken shell eggs above the heating elements as the bubbles rise to the top surface of the water in the bath;
a batch carrier arrangement that holds batches of shell eggs in the bath and includes an advance motor that moves the batch carrier arrangement and thereby the respective batches of shell eggs through and between stage locations in the water bath; and
a batch processing control system is programmed with at least one pasteurization protocol based on an empirically verified D-value pasteurization model predicting reduction of Salmonella Enteritidis in the yolk of chicken shell eggs held at an unrefrigerated temperature prior to pasteurization, wherein the empirically verified D-value pasteurization model is determined by collecting thermal validation data on kill levels of Salmonella Enteritidis in chicken shell eggs that are inoculated, held at an unrefrigerated temperature prior to pasteurization and subsequently heated to suitable pasteurization temperatures, said protocol setting the temperature set point value for the water bath and setting a total dwell time in the bath for each batch to a predetermined time sufficient to ensure that the yolk of the egg have been pasteurized to achieve at least a statistical 5-log reduction of Salmonella Enteritidis that may have been present in the yolk in its unpasteurized form according to said D-value pasteurization model.

15. The system of claim 14 wherein the total dwell time is calculated so that the chicken eggs are not removed from the water bath until the eggs have been in the water bath for an amount of time sufficient to achieve a statistical 5-log reduction of Salmonella Enteritidis that may have been present in the yolk according to said D-value pasteurization model.

16. The system of claim 14 wherein the total dwell time is calculated to enable removal of the chicken eggs from the heated water bath prior to achieving a statistical 5-logreduction of Salmonella Enteritidis that may have been present in the yolk of the eggs prior to pasteurization according to said D-value pasteurization model, wherein pasteurization continues after the egg is removed from that heated water bath until the yolk cools below 128° F. and the dwell time in the water bath is sufficient to achieve said 5-log reduction before the yolk cools to below 128° F.

17. The system of claim 14 wherein the unrefrigerated temperature prior to the pasteurization when collecting thermal validation data to determine the empirically verified D-value pasteurization model for the statistical reduction of Salmonella Enteritidis in the yolk of chicken shell eggs is substantially about 65° F.

18. The system of claim 14 wherein the determination of the empirically verified D-value pasteurization model comprises inoculating chicken shell eggs with a Salmonella cocktail including at least one strains of Salmonella Enteritidis.

19. The system of claim 14 wherein the determination of the empirically verified D-value pasteurization model comprises inoculating chicken shell eggs with a Salmonella cocktail including strains of: Salmonella Enteritidis, Salmonella Typhimurium, Salmonella Heidelberg, and Salmonella Othmarschen.

20. The system of claim 14 wherein said one pasteurization protocol is for an unrefrigerated, Large chicken egg, the temperature set point for the water bath is 133° F.+/−0.5° F., and the predetermined dwell time is no more than 50 minutes.

21. The system of claim 14 wherein said one pasteurization protocol is for an unrefrigerated, Large chicken egg, the temperature set point for the water bath is 134° F.+/−0.5° F., and the predetermined dwell time is no more than 45 minutes.

22. The shell egg pasteurization system as recited in claim 14 further comprising a cooling system that operates to selectively lower the temperature of the water in the pasteurization bath when the temperature in the bath approaches an upper temperature limit that is set higher than the temperature set point value.
